(19) United States
(12) Patent Application Publication
(10) Pub. No.: US 2022/0041591 A1
(43) Pub. Date: Feb. 10, 2022

(54) COMPOUNDS FOR TREATING DIABETES AND/OR RELATED CONDITIONS

(71) Applicants: AUCKLAND UNISERVICES LIMITED, Auckland (NZ); JINAN UNIVERSITY, Guangzhou (CN)

(72) Inventors: Jacqueline F. AITKEN, Auckland (NZ); Garth James Smith COOPER, Auckland (NZ); Shaoping ZHANG, Auckland (NZ); Ke DING, Guangzhou (CN); Zhengqiu LI, Guangzhou (CN); Binbin ZHENG, Guangzhou (CN)

(21) Appl. No.: 17/286,794

(22) PCT Filed: Oct. 18, 2019
(86) PCT No.: PCT/NZ2019/050138

§ 371 Date: Apr. 19, 2021

(30) Foreign Application Priority Data
Oct. 19, 2018 (CN) 201811226113.8
Dec. 11, 2018 (NZ) 749140

(51) Int. Cl. C07D 471/04; C07D 213/75; A61P 1/18

(52) U.S. Cl. CPC C07D 471/04; A61P 1/18; C07D 213/75

(57) ABSTRACT

Compounds useful for treating or preventing diabetes and related conditions, and for preventing, inhibiting or reversing amylin-amyloid fibril formation, preventing islets of Langerhans (Beta)-cell death, preventing or reversing the transition from soluble human amylin to insoluble human amylin, preventing and inhibiting or reversing cytotoxic amylin fibril formation. The invention relates to compounds of Formula I and compounds of Formula II:

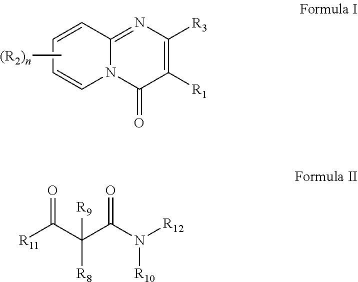

### FIELD OF THE INVENTION

The present invention relates to compounds useful for treating or preventing diabetes and related conditions, and for preventing, inhibiting or reversing amylin-amyloid fibril formation, preventing islets of Langerhans (Beta)-cell death, preventing or reversing the transition from soluble human amylin to insoluble human amylin, preventing and inhibiting or reversing cytotoxic amylin fibril formation. The present invention also relates to pharmaceutical compositions comprising compounds of the invention, uses of the compounds for the above-mentioned purposes, and their use in the manufacture of medicaments for the above-mentioned purposes.

### BACKGROUND OF THE INVENTION

Misfolded protein aggregates known as amyloids have been reported to play a key role in the pathology of a number of diseases, such as rheumatoid arthritis, atherosclerosis, Alzheimer's disease, Parkinson's disease, Huntington's disease, and diabetes.

Misfolded human amylin (hA) is the main component of islet amylin amyloid in diabetic patients and is thought to be at least partly responsible for the development and progression of type II diabetes mellitus (type II diabetes). Human amylin therefore represents a potential target for developing medicines that can prevent or slow the progression of type II diabetes.

Some molecules capable of altering hA-misfolding and aggregation are already known in the art, and include the broad-spectrum antibiotic, tetracycline. However, many of these molecules suffer from disadvantages, for example side-effect profiles or off-target activities, which make them unsuitable for long-term use. There remains a need for compounds capable of preventing, inhibiting and or reversing amylin-amyloid fibril formation, and/or of preventing islets of Langerhans Beta-cell death, and/or of preventing and/or reversing the transition from soluble human amylin to insoluble human amylin, and/or of preventing and inhibiting and/or reversing cytotoxic amylin fibril formation.

It is an object of the present invention to provide compounds which overcome or at least partially ameliorate some of the abovementioned disadvantages and/or which at least provide the public with a useful choice.

Other objects of the invention may become apparent from the following description which is given by way of example only.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### SUMMARY OF THE INVENTION

In one aspect the invention provides a compound of Formula I:

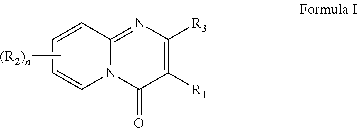

wherein:
R₁ is selected from hydrogen, halide, optionally substituted C₁₋₄alkyl, and optionally substituted C₂₋₄alkenyl;
each R₂ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₃ is selected from halide, optionally substituted aryl, optionally substituted heteroaryl, optionally substituted heterocycloalkyl, and optionally substituted cycloalkyl;
n is selected from 0, 1, 2, 3 and 4;
or a pharmaceutically acceptable salt thereof.

In one aspect the invention provides a compound of Formula II:

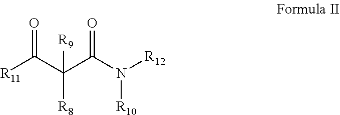

wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
R₁₁ is selected from optionally substituted aryl, optionally substituted heteroaryl, optionally substituted cycloalkyl, and optionally substituted heterocycloalkyl;
R₁₂ is selected from optionally substituted aryl, optionally substituted heteroaryl, optionally substituted cycloalkyl, and optionally substituted heterocycloalkyl;
or a pharmaceutically acceptable salt thereof.

In another aspect the invention relates to a composition comprising a compound of Formula I or Formula II, together with a carrier, excipient or diluent.

In another aspect, the invention relates to a method of synthesising a compound of Formula I or Formula II as depicted or described herein, for example as depicted or described in the Examples.

In a further aspect the invention relates to a method of treating or preventing an amylin amyloid-associated disease, the method comprising administering to a subject in need thereof an effective amount of a compound of Formula I or Formula II or a pharmaceutically acceptable salt thereof.

In a further aspect the invention relates to a method of inhibiting, preventing, or reversing an amylin amyloidosis or the formation of amylin-amyloid fibrils or amylin amyloid plaques in a subject in need thereof, the method comprising administering to a subject in need thereof an effective amount of a compound of Formula I or Formula II or a pharmaceutically acceptable salt thereof.

In a further aspect the invention relates to a method of inhibiting, preventing, or reversing an amylin amyloidosis, or the formation of one or more amylin-amyloid fibrils or amylin amyloid plaques, the method comprising contacting the amylin amyloidosis or one or more amylin-amyloid fibrils or amylin amyloid plaques with an effective amount of a compound of Formula I or Formula II or a pharmaceutically acceptable salt thereof.

In one embodiment, the method is a method of inhibiting, preventing, or reversing the formation of islet amylin-amyloid fibrils.

In another aspect the invention relates to a method of treating or preventing diabetes in a subject in need thereof, the method comprising administering to a subject in need thereof an effective amount of a compound of Formula I or Formula II or a pharmaceutically acceptable salt thereof.

In another aspect the invention relates to a method of treating or preventing islet of Langerhans beta-cell death in a subject in need thereof, the method comprising administering to a subject in need thereof an effective amount of a compound of Formula I or Formula II or a pharmaceutically acceptable salt thereof.

In a further aspect the invention relates to a method of treating or preventing islet of Langerhans beta-cell death, the method comprising contacting one or more islet of Langerhans beta-cells with an effective amount of a compound of Formula I or Formula II or a pharmaceutically acceptable salt thereof.

In another aspect, the invention provides a pharmaceutical composition comprising one or more compounds of Formula I or Formula II, optionally together with a pharmaceutically acceptable carrier or excipient.

The invention further relates to a compound of Formula I or Formula II for use in one or more of treating or preventing an amylin amyloid-associated disease, inhibiting, preventing, or reversing an amylin amyloidosis or the formation of amylin-amyloid fibrils or amylin amyloid plaques, inhibiting, preventing, or reversing the formation of islet amylin-amyloid fibrils, treating or preventing diabetes, or treating or preventing islet of Langerhans beta-cell death, for example in a subject in need thereof.

The invention additionally relates to the use of a compound of Formula I or Formula II in the preparation of a medicament for one or more of treating or preventing an amylin amyloid-associated disease, inhibiting, preventing, or reversing an amylin amyloidosis or the formation of amylin-amyloid fibrils or amylin amyloid plaques, inhibiting, preventing, or reversing the formation of islet amylin-amyloid fibrils, treating or preventing diabetes, or treating or preventing islet of Langerhans beta-cell death, for example in a subject in need thereof.

The embodiments set out herein may relate to any of the above aspects.

Other aspects of the invention, which are not limited to or by the information in this Summary of the Invention, may become apparent from the following description which is given by way of example only and with reference to the accompanying figures.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### BRIEF DESCRIPTION OF THE FIGURES

FIG. 1 is three graphs showing the results of thioflavin-T assays investigating the ability of compounds described herein to inhibit amylin fibril formation, as discussed in Example 12. FIG. 1A presents the results at hA:compound molar ratios of 1:1, FIG. 1B presents the results at hA:compound molar ratios of 1:0.1, and FIG. 1C presents the results at hA:compound molar ratios of 1:0.01.

FIG. 2 is a graph showing the results of cell death assays investigating the ability of compounds described herein to inhibit amylin-evoked cell death, as discussed in Example 13.

FIG. 3 is a graph showing the results of thioflavin-T assays investigating the ability of compounds described herein to inhibit amylin fibril formation, as discussed in Example 14.

FIG. 4 is three graphs showing the results of thioflavin-T assays investigating the ability of compounds described herein to inhibit amylin fibril formation, as discussed in Example 14. FIG. 4A presents the results at hA:compound molar ratios of 1:1, FIG. 4B presents the results at hA:compound molar ratios of 1:0.1, and FIG. 4C presents the results at hA:compound molar ratios of 1:0.01.

FIG. 5 is two graphs showing the results of cell death assays investigating the ability of compounds described herein to inhibit amylin-evoked cell death, as discussed in Example 15. FIG. 5A presents data observed in CM cells, while FIG. 5B presents data observed in RINm5F cells.

FIG. 6 is two graphs showing the results of cell death assays investigating the ability of compounds described herein to inhibit amylin-evoked cell death, as discussed in Example 16. FIG. 6A presents data observed in CM cells, while FIG. 6B presents data observed in RINm5F cells.

FIG. 7 is two graphs showing the results of cell death assays investigating the ability of compounds described herein to inhibit amylin-evoked cell death, as discussed in Example 17. FIG. 7A presents data observed in CM cells, while FIG. 7B presents data observed in RINm5F cells.

### DETAILED DESCRIPTION

The present invention provides various compounds suitable for use in the treatment of amylin amyloidoses, including, for example, diabetes. The compounds have advantageous physicochemical and/or therapeutic properties rendering them particularly suitable for use in the treatment of amylin amyloid-associated diseases, such as diabetes.

In certain embodiments, the compound of Formula I is a compound of Formula Ia:

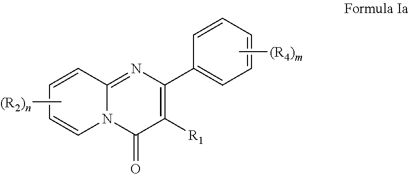

wherein
R₁ is selected from hydrogen, optionally substituted C₁₋₄alkyl, and optionally substituted C₂₋₄alkenyl;
each R₂ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
each R₄ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
n is selected from 0, 1, 2, 3 and 4;
m is selected from 0, 1, 2, 3, 4 and 5;
or a pharmaceutically acceptable salt thereof.

In certain embodiments, the compound of Formula I is a compound of Formula Ib:

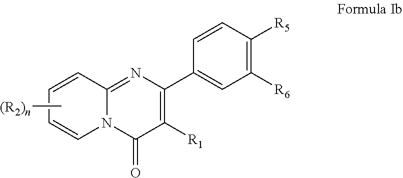

wherein:
R₁ is selected from hydrogen, optionally substituted C₁₋₄alkyl, and optionally substituted C₂₋₄alkyenyl;
each R₂ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₅ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₆ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
n is selected from 0, 1, 2, 3 and 4
or a pharmaceutically acceptable salt thereof.

In certain embodiments, the compound of Formula I is a compound of Formula Ic:

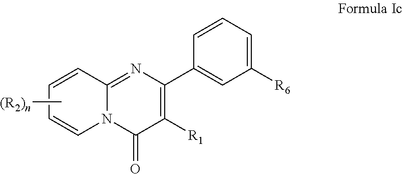

wherein
R₁ is selected from hydrogen, optionally substituted C₁₋₄alkyl, and optionally substituted C₂₋₄alkyenyl;
each R₂ is independently selected from hydroxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₆ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
n is selected from 0, 1, 2, 3 and 4;
or a pharmaceutically acceptable salt thereof.

In certain embodiments, the compound of Formula I is a compound of Formula Id:

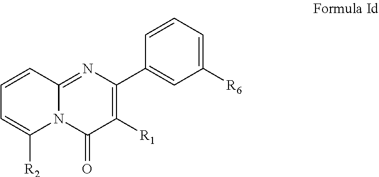

wherein
R₁ is selected from hydrogen, optionally substituted C₁₋₄alkyl, and optionally substituted C₂₋₄alkenyl;
R₂ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₆ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
or a pharmaceutically acceptable salt thereof.

In compounds of any one of Formulae I, Ia, Ib, Ic, or Id:
R₁ is hydrogen;
each R₂ is independently selected from hydroxy and alkyloxy, such as methoxy;
R₃ is selected from optionally substituted aryl;
each R₄ is independently selected from hydroxy and alkyloxy, such as methoxy;
R₅ is selected from hydroxy and alkyloxy, such as methoxy;
R₆ is selected from hydroxy and alkyloxy, such as methoxy;
n is 1; and/or
m is 1 or 2.

In certain embodiments, the compound of Formula I is selected from compounds 2, 5, 6 and 8 or a pharmaceutically acceptable salt thereof.

In certain embodiments, the compound of Formula II is a compound of Formula IIa:

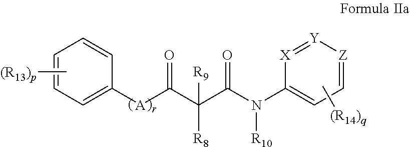

wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
each R₁₃ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
each R₁₄ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
A is selected from

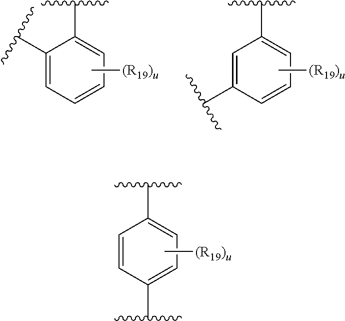

wherein each R₁₉ is independently selected from hydroxy, halide, carboxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl; and
u is selected from 0, 1, 2, 3 and 4;
X is selected from C and N;
Y is selected from C and N;
Z is selected from C and N;
p is selected from 0, 1, 2, 3, 4 and 5;
q is selected from 0, 1, 2, 3 and 4;
r is selected from 0 and 1;
or a pharmaceutically acceptable salt thereof.

In certain embodiments, the compound of Formula II is a compound of Formula IIb:

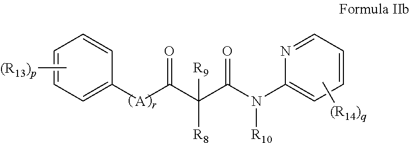

wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
each R₁₃ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
each R₁₄ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
A is selected from

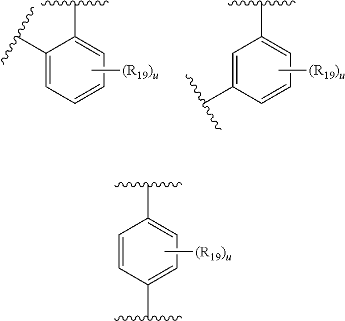

wherein each R₁₉ is independently selected from hydroxy, halide, carboxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl; and
u is selected from 0, 1, 2, 3 and 4;
p is selected from 0, 1, 2, 3, 4 and 5;
q is selected from 0, 1, 2, 3 and 4;
r is selected from 0 and 1;
or a pharmaceutically acceptable salt thereof.

In certain embodiments, the compound of Formula II is a compound of Formula IIc:

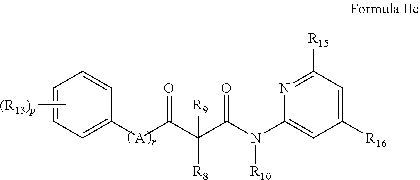

wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
each R₁₃ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₅ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₆ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
A is selected from

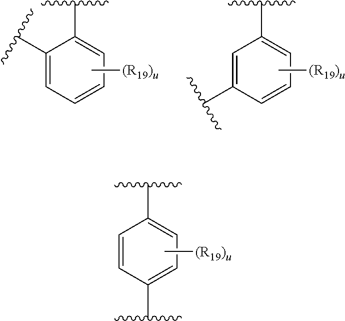

wherein the or each R₁₉ is independently selected from hydroxy, halide, carboxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl; and
u is selected from 0, 1, 2, 3 and 4;
p is selected from 0, 1, 2, 3, 4 and 5;
r is selected from 0 and 1;
or a pharmaceutically acceptable salt thereof.

In certain embodiments, the compound of Formula II is a compound of Formula IId:

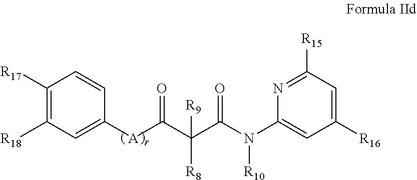

wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
R₁₅ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₆ is selected from hydroxy, halide, optionally substituted amino, optionally substituted aminoalkyl, optionally substituted aminoalkenyl, aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₇ is selected from hydroxy, halide, carboxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₅ is selected from hydroxy, halide, carboxy, optionally substituted amino, optionally substituted aminoalkyl, optionally substituted aminoalkenyl, aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
A is selected from

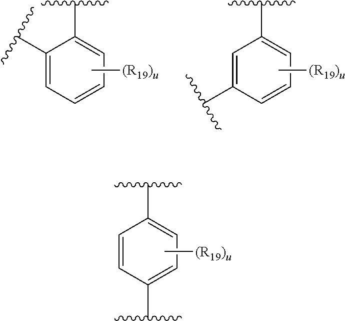

wherein each R₁₉ is independently selected from hydroxy, carboxy, optionally substituted amino, optionally substituted aminoalkyl, optionally substituted aminoalkenyl, aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl; and
u is selected from 0, 1, 2, 3 and 4;
r is selected from 0 and 1;
or a pharmaceutically acceptable salt thereof.

In compounds of any one of Formulae II, IIa, IIb, IIc, or IId:
R₈ is hydrogen;
R₉ is hydrogen;
R₁₀ is hydrogen;
R₁₁ is optionally substituted arylaryl, such as optionally biphenylyl;
R₁₂ is optionally substituted heteroaryl, such as optionally substituted pyridyl;
R₁₃ is carboxy, alkyloxy (such as methoxy), hydroxy or halide;
R₁₄ is alkyloxy (such as methoxy) or halide;
A is phen-1,4-yl;
u is 0;
X is N, and Y and Z are C;
R₁₅ is alkyloxy (such as methoxy) or halide;
R₁₆ is alkyloxy (such as methoxy) or halide;
R₁₇ is carboxy, alkyloxy (such as methoxy), hydroxy or halide; and/or
R₁₅ is carboxy, alkyloxy (such as methoxy), hydroxy or halide.

In one embodiment, the compound of Formula II is selected from compounds 1, 3, 4, 9, 10, 24 and 26 or a pharmaceutically acceptable salt thereof.

Compounds of the invention include, but are not limited to, the following:

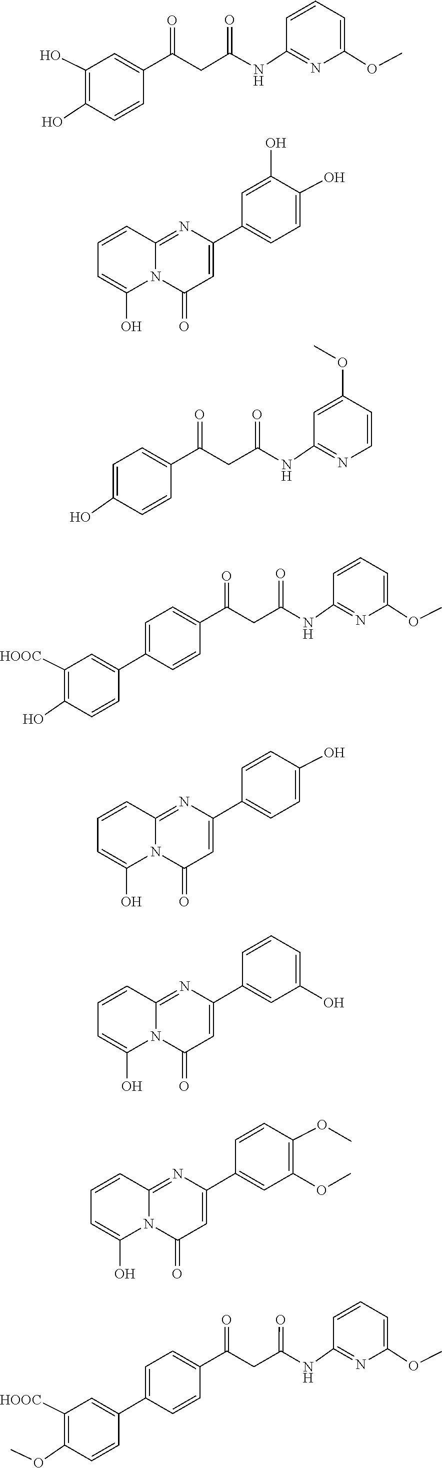

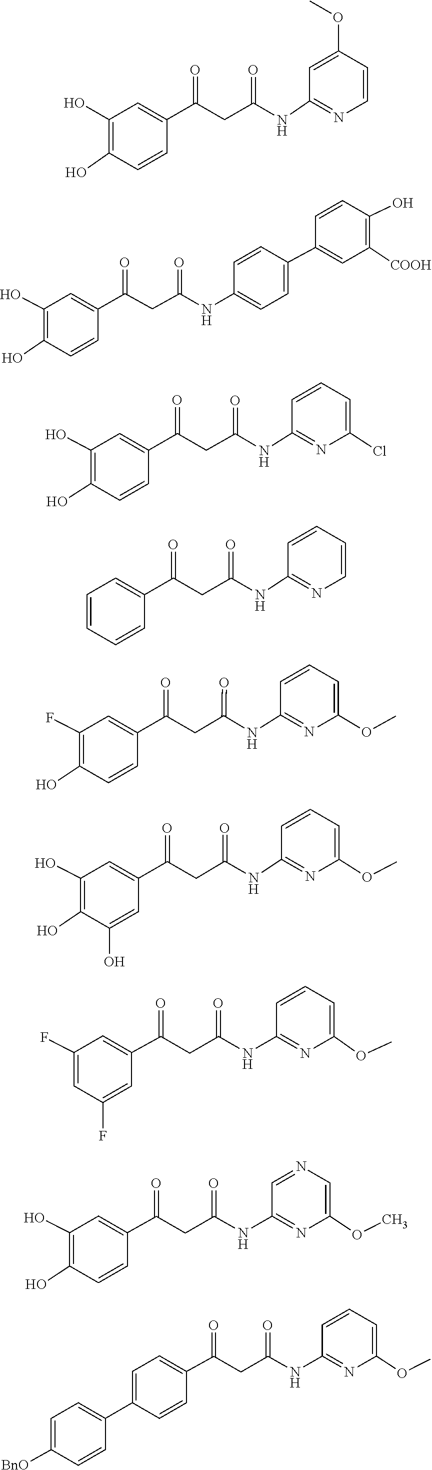

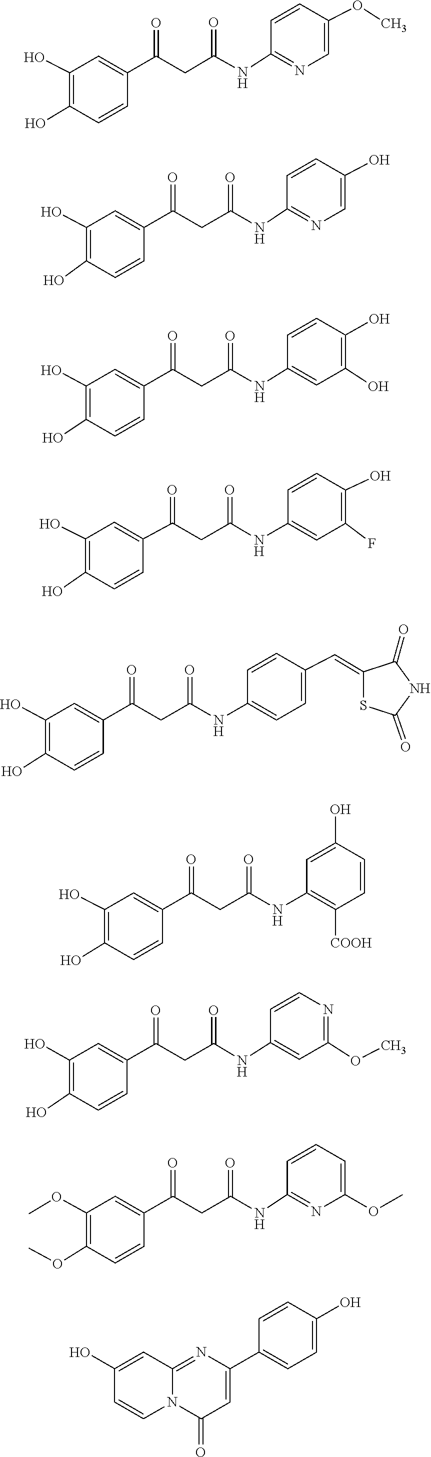

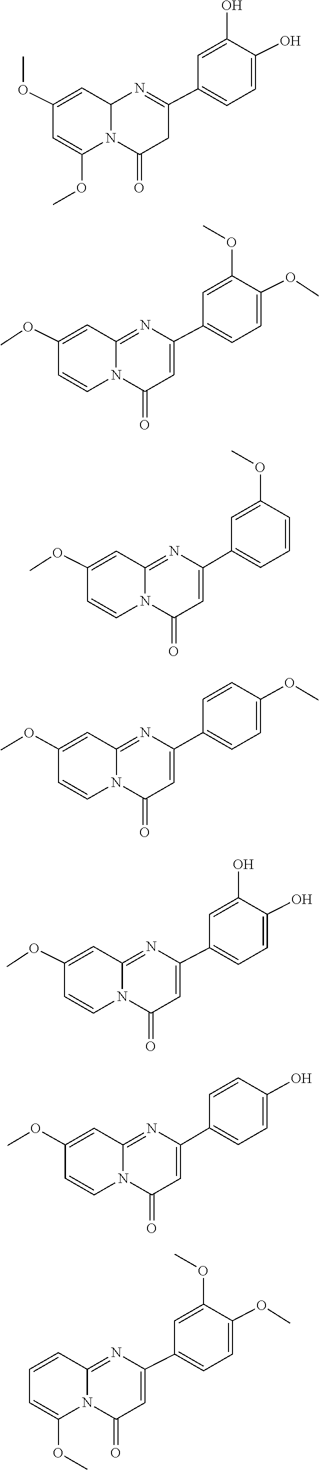

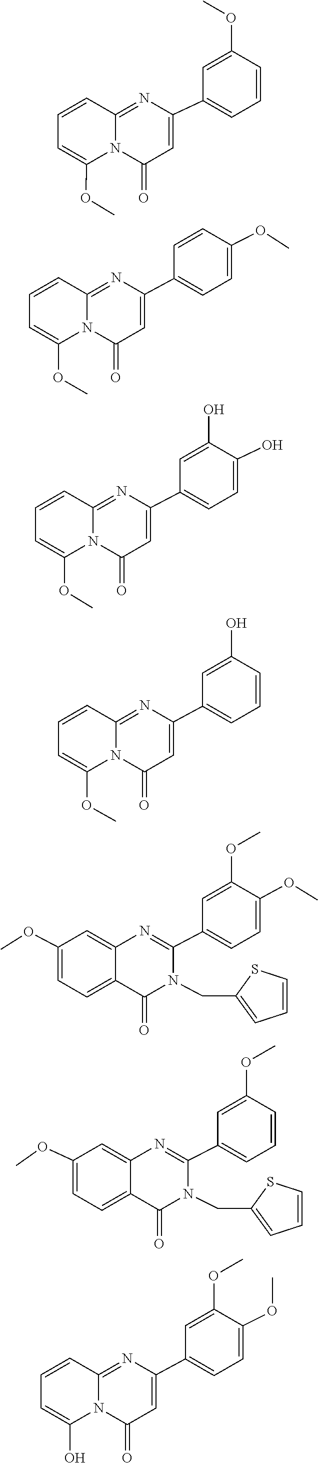

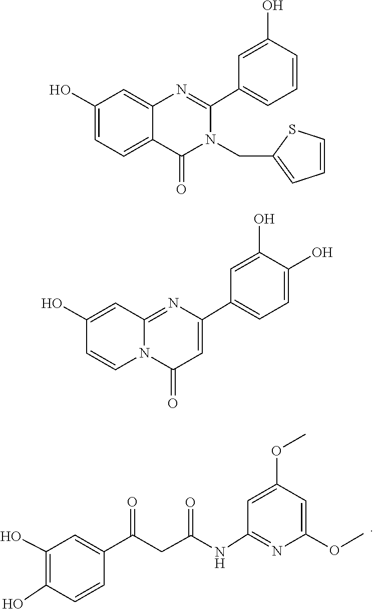 [SMILES: O=c1c2ccc(O)cc2nc(-c2cccc(O)c2)n1Cc1cccs1 ; O=c1cc(-c2ccc(O)c(O)c2)nc2cc(O)ccn12 ; COc1cc(NC(=O)CC(=O)c2ccc(O)c(O)c2)nc(OC)c1]

As used herein the term “and/or” means “and” or “or”, or both.

As used herein “(s)” following a noun means the plural and/or singular forms of the noun.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term “comprising” as used in this specification means “consisting at least in part of”. When interpreting statements in this specification which include that term, the features, prefaced by that term in each statement or claim, all need to be present but other features can also be present. Related terms such as “comprise” and “comprised” are to be interpreted in the same manner.

### Human Amylin (hA)

The present invention relates in certain embodiments to compounds capable of preventing or reversing human amylin (hA) misfolding, aggregation and/or amylin plaque amyloid formation. In certain embodiments, the present invention relates to compounds capable of preventing and/or reversing the transition from soluble human amylin to insoluble human amylin, and/or of preventing and inhibiting and/or reversing cytotoxic amylin fibril formation.

Human amylin, also known as islet amylin amyloid polypeptide (IAPP), is a 37-residue peptide hormone produced in and secreted by the beta-cells of the islets of Langerhans in the human pancreas. The production of hA in the body involves the conversion of an 89-residue coding sequence into a 67 amino acid residue proamylin sequence (proIAPP). This proamylin sequence then undergoes post-translational modification to produce hA. Human amylin is produced under normal conditions in the body and, together with insulin, plays a role in glycemic control.

### Amylin Amyloidosis

The term “amyloidosis” as used herein refers to the deposition of amyloid in the body. It will be apparent to a person skilled in the art that there is usually a relationship between the identity of the protein and where in the body an amyloidosis manifests. Consequently, several types of amyloids exist, each of which is classified depending on the protein it comprises, and specific types of amyloidoses are classified based on the site of amyloid accumulation.

The deposition of amyloids in the body often involves the accumulation of insoluble amyloid fibrils that comprise misfolded proteins. The terms “amyloid”, “amyloid fibril” and “amyloid plaque” as used herein refer to protein aggregates resulting from the misfolding of proteins into forms that facilitate and/or promote aggregation, and/or that may lead to cytotoxicity.

Human amylin is one protein that may misfold leading to the conversion of soluble amylin monomers and soluble amylin oligomers into insoluble amylin-amyloid fibrils. The terms “amylin amyloid”, “amylin-amyloid fibril”, “amylin aggregate” or “amylin amyloid plaque” are used herein to refer to amylin amyloids comprising human amylin as the protein component in an insoluble state. The terms “islet amylin amyloids”, “islet amylin-amyloid fibrils”, “islet aggregates” or “islet amylin amyloid plaques” are used herein to refer to amylin amyloids comprising human amylin as the protein component in an insoluble state and typically found in the islets of Langehans. When present in the islets, these insoluble aggregates may lead to beta-cell death and other cytotoxic effects, the mechanisms of which are poorly understood. Without wishing to be bound by theory the inventors believe that aggregation of islet amylin amyloids into islet amylin-amyloid fibrils and/or hA misfolding is at least partly responsible for the toxicity that is observed in, and that is thought to play a role in the pathology of amylin amyloid-associated diseases, for example, type II diabetes.

### Disruption of Amylin Amyloids

The inventors have found that the compounds described herein are capable of disrupting amylin amyloid formation, for example islet amylin amyloid formation.

The term “disruption” as used herein refers to both priori disruption and posteriori disruption of amylin amyloidosis. Priori disruption refers to the interference of the compounds described herein with any part of the process involving the conversion of soluble monomers and oligomers, such as amylin monomers and oligomers, into insoluble amylin amyloids. Posteriori disruption as used herein refers to the disaggregation of insoluble amylin amyloids that are already formed, for example islet amylin amyloids, which may include the re-solubilisation of the constituent protein.

Disruption of insoluble amylin amyloids may occur directly or indirectly. Direct disruption involves one or more compounds described herein or a metabolite thereof binding to a part of the protein monomers or oligomers, such as human amylin monomers or oligomers, or part of a pre-formed aggregate, and physically preventing further aggregation and/or reversing aggregation, for example by weakening interactions between the monomers and/or oligomers in the aggregate. Indirect disruption, in contrast, involves preventing fibril or aggregate formation via mechanisms that do not require direct contact between the compound and the protein, for example, by altering local conditions to favour a soluble form of the protein over an insoluble form, or by favouring a non-aggregating conformation of the monomers or oligomers.

The term “amylin monomer” as used herein refers to 37-residue peptide hormone produced in and secreted by the Beta-cells of the islets of Langerhans. The amino acid sequence for human preprotein, also referred to as islet amyloid polypeptide preprotein, is present at RefSeq NP_000406.1, and at UniProtKB/SwissProt A0A024RAU1.

The term “oligomer” as used herein refers to a molecule with two or more monomer units. Amylin oligomers may comprise from about 2, 3, 4, 5, 6, 7, 8, 10, 15, 16, 20 or 25 monomer units to about 100 monomer units, or from about 100 to about 1000, 2000, 3000, or more monomer units. The term “amylin oligomer” refers to oligomers in which the monomer units are human amylin.

The term “interfere” and “interference” as used herein in the context of protein aggregation refers to the disruption of the process of aggregation by a compound described herein, such that aggregation is slowed or prevented.

In some embodiments direct disruption of aggregates, such as islet aggregates, by the compounds described herein involves binding via covalent interactions, and in other embodiments involves binding via non-covalent interactions. In various embodiments the compounds described herein interact with the monomers or with oligomers of the proteins making up the amylin amyloids or to the amylin amyloids themselves, by a combination of one or more covalent interactions and/or one or more non-covalent interactions, for example van der Waals interactions.

For example, without wishing to be bound by theory the inventors believe that in various embodiments the compounds described herein bind covalently to the monomeric forms of proteins, for example hA. This is believed, again without wishing to be bound by any theory, to stabilize the conformation of monomeric forms and/or soluble oligomers, non-toxic forms, inhibit amylin amyloid formation directly.

Disruption of amylin amyloids may also occur indirectly, for example if the compounds do not bind directly to monomers or oligomers of proteins making up the amylin amyloids, or to amylin amyloids themselves, but affect another part of the amylin amyloidosis process that prevents or inhibits the formation of further aggregates or leads to the disaggregation of pre-formed aggregates.

Without wishing to be bound by theory the inventors believe that one mechanism for the formation of insoluble islet amylin amyloids begins with the aggregation of proamylin, which then serves as an initiation site for the deposition of insoluble islet amylin-amyloid fibrils. In some embodiments disruption of islet amylin amyloids thus occurs by directly or indirectly disrupting the aggregation of or on proamylin.

In the case of islet amylin amyloids, experiments report that under normal conditions hA may be reasonably unstructured and made up mostly of random coil structures. Under certain conditions often associated with pathogenesis, hA is expected to misfold to produce Beta-sheets that stack to form highly structured aggregates comprising layers of Beta-sheets. In some embodiments disruption of islet amylin amyloids comprises the prevention or reversal of hA misfolding. Misfolding of the protein components of amylin amyloids is also thought to play a role in other diseases associated with amylin amyloidosis.

In various embodiments the compounds described herein disrupt amylin amyloid aggregates by one or more mechanisms selected from the group consisting of preventing, inhibiting and/or reversing amylin-amyloid fibril formation, preventing and/or reversing the transition from soluble human amylin to insoluble human amylin, and preventing, inhibiting and/or reversing cytotoxic amylin fibril formation. In some embodiments the compounds described herein disrupt amylin-amyloid fibril formation by preventing, inhibiting and/or reversing protein misfolding. For example, the compounds described herein disrupt islet amylin-amyloid fibril formation by preventing, reversing and/or reversing hA misfolding.

In various embodiments the compounds described herein prevent, inhibit and/or reverse protein misfolding and/or prevent, inhibit and/or reverse protein aggregation.

The terms “aggregation” or “aggregate” as used herein refer to the accumulation of protein, such as human amylin or its precursor proIAPP, particularly in insoluble forms. Without wishing to be bound by theory, the inventors believe that the aggregation potential of hA is promoted by the misfolding of amylin into structured Beta-sheets.

### Effect on Disease States

Amylin amyloids are thought to play a role in the pathogenesis of a number of diseases. The term “amylin amyloid-associated disease” as used herein refers to diseases including but not limited to diabetes, including type II diabetes (T2D), metabolic syndrome, syndrome X, dysregulation of blood glucose, insulin resistance, and the like.

Amylin amyloid-associated diseases occur in a number of animals, including mammals, for example human beings.

The inventors have found that the compounds described herein are able to prevent, inhibit and/or reverse, for example, amylin-amyloid fibril formation, islet of Langerhans Beta-cell death, the transition from soluble human amylin to insoluble human amylin and cytotoxic oligomer formation. Without wishing to be bound by theory, the inventors believe that these processes are associated with the development and/or progression of one or more amylin amyloid-associated diseases, such as, for example, type II diabetes.

The term “prevent” as used herein refers to the halting of a process, for example amylin-amyloid fibril formation, islet of Langerhans beta-cell death, the transition from soluble human amylin to insoluble human amylin, and cytotoxic oligomer formation that has not yet begun. In certain embodiments, such prevention is for a certain period of time—for example, for so long as the concentration of the compound or compounds as described herein, such as the concentration local to the hA, is maintained above a certain threshold. It will be appreciated that in such embodiments the term “prevent” does not contemplate prevention in perpetuity.

The term “inhibit” as used herein is used in a similar manner to “prevent” but refers to the halting of a process, for example amylin-amyloid fibril formation, islet of Langerhans Beta-cell death, the transition from soluble human amylin to insoluble human amylin and cytotoxic oligomer formation, that has already begun.

The prevention, inhibition or reversal of processes associated with the development of one or more amylin amyloid-associated diseases may manifest in a number of ways. For example, in some embodiments the prevention, inhibition or reversal of processes such as for example amylin-amyloid fibril formation, islet of Langerhans Beta-cell death, the transition from soluble human amylin to insoluble human amylin and cytotoxic oligomer formation, leads to slowing of disease progression and improved quality of life in subjects with the disease.

In various embodiments the prevention, inhibition or reversal of processes associated with the development of one or more amylin amyloid-associated diseases manifests as an increased rate of survival. For example, prevention, inhibition or reversal of a process associated with hA amylin amyloid formation will in certain embodiments manifest as an increased rate of survival of islet Beta-cells, for example.

The term “treat” as used herein refers to inhibiting or arresting the development of an amylin amyloid-associated disease and/or causing the reduction, remission or regression of an amylin amyloid-associated disease or one or more side effects thereof. Methods of assessing treatment, including methods of assessing inhibition, arrest, reduction, remission and/or regression of disease states are known and will be apparent to a person skilled in the art.

The term “reverse” as used herein refers to the return of an amylin amyloid-associated disease to a former or less developed state. The terms “remission” and “regression” are to be interpreted in a similar manner.

### Administration and Formulation

The term “administering” as used herein refers to providing a therapeutically effective amount of a compound to a subject using one or more methods of administering compounds known in the art. These methods comprise administering compounds using oral, sublingual, intravenous, subcutaneous, transcutaneous, intramuscular, intracutaneous, intrathecal, epidural, intraocular, intracranial, inhalation, rectal, vaginal, and the like administration.

In exemplary embodiments one or more active agents may be administered orally.

It will be apparent to a person skilled in the art that the formulation of the compounds described herein will depend on the method of administration. For example, the compounds described herein are in certain embodiments formulated as creams, lotions, tablets, capsules, pellets, dispersible powders, granules, suppositories, syrups, elixirs, lozenges, injectable solutions, sterile aqueous or non-aqueous solutions, suspension or emulsions, patches and the like.

For example in various embodiments the compounds described herein are formulated as tablets, capsules, pellets, dispersible powders, granules, syrups, suspensions or emulsions.

In exemplary embodiments the compounds described herein formulated as a solid dosage form, such as tablets, capsules or pellets.

The formulations that are suitable for a particular method of administration will be apparent to those skilled in the art.

As will be readily appreciated by those skilled in the art, the route of administration and the nature of the pharmaceutically acceptable carrier will depend on the nature of the condition and the mammal to be treated. It is believed that the choice of a particular carrier or delivery system, and route of administration could be readily determined by a person skilled in the art. In the preparation of any formulation containing the compound actives care should be taken to ensure that the activity of the compound is not destroyed in the process and that the compound is able to reach its site of action without being destroyed. In some circumstances it may be necessary to protect the compound by means known in the art, such as, for example, microencapsulation. Similarly, the route of administration chosen should be such that the compound reaches its site of action.

Those skilled in the art may readily determine appropriate formulations for the compounds of the present invention using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, reducing agents such as methionine or sulphite, and metal chelators such as EDTA.

The compound as hereinbefore described, or pharmaceutically acceptable salt thereof, may be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for compound actives, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolality, for example, sugars or sodium chloride. In various embodiments, the formulation for injection will be isotonic with blood. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection or infusion.

Sterile injectable solutions are prepared by incorporating the active compound in the required amount in the appropriate solvent with various of the other ingredients such as those enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

Other pharmaceutical forms include oral and enteral formulations of the present invention, in which the active compound may be formulated with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal or sublingual tablets, troches, capsules, elixirs, suspensions, syrups, wafers, and the like. The amount of active compound in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations, including those that allow specific delivery of the active compound to specific regions of the gut.

Liquid formulations may also be administered enterally via a stomach or oesophageal tube.

Enteral formulations may be prepared in the form of suppositories by mixing with appropriate bases, such as emulsifying bases or water-soluble bases. It is also possible, but not necessary, for the compounds of the present invention to be administered topically, intranasally, intravaginally, intraocularly and the like.

The present invention also extends to any other forms suitable for administration, for example topical application such as creams, lotions and gels, or compositions suitable for inhalation or intranasal delivery, for example solutions, dry powders, suspensions or emulsions.

The compounds of the present invention may be administered by inhalation in the form of an aerosol spray from a pressurised dispenser or container, which contains a propellant such as carbon dioxide gas, dichlorodifluoromethane, nitrogen, propane or other suitable gas or combination of gases. The compounds may also be administered using a nebuliser.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the invention are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired as herein disclosed in detail.

As mentioned above the principal active ingredient may be compounded for convenient and effective administration in therapeutically effective amounts with a suitable pharmaceutically acceptable vehicle in dosage unit form. A unit dosage form can, for example, contain the principal active compound in amounts ranging from 0.25 μg to about 2000 mg. Expressed in proportions, the active compound may be present in from about 0.25 μg to about 2000 mg/mL of carrier. In the case of compositions containing supplementary active ingredients, the dosages are determined by reference to the usual dose and manner of administration of the said ingredients.

The term “therapeutically effective amount” as used herein refers to a dose of a compound sufficient to provide a concentration high enough to effect the desired result. For example, in certain embodiments a therapeutically effect amount is a dose of a compound described herein sufficient to result in one or more of the following; the prevention, inhibition or reversal of amylin-amyloid fibril formation, islet of Langerhans beta-cell death, the transition from soluble human amylin to insoluble human amylin, and cytotoxic oligomer formation.

The therapeutically effective amount of a compound will in certain embodiments be affected by a number of factors, and can be adjusted based on these factors. For example, the therapeutically effective dose may be affected by the bodyweight of the subject, metabolic capacity and synergy between combinations of actives administered. The dose that can be administered to a subject may also be affected by other factors such as interactions with other medicines that the subject is taking and severity of/ability to tolerate any side effects of the compounds administered.

In some embodiments, the desired result to be achieved by the therapeutically effective amount comprises the slowing of disease progression, an improvement in the quality of life of the subject and/or an increased rate of survival.

In various embodiments, the desired result to be achieved by the therapeutically effective amount is the amelioration of one or more symptoms associated with an amylin amyloid-associated disease, for example reduction in loss of kidney function, heart failure, obstructive sleep apnea, difficulty swallowing or synovitis.

In various embodiments, the desired result to be achieved by the therapeutically effective amount is the amelioration of one or more symptoms associated with type II diabetes, for example weight loss, polyuria, polydipsia, polyphagia, blurry vision, headache, fatigue or diabetic dermadromes, and signs such as lowering of blood glucose levels and HbA1C

Where the compound comprises one or more functional groups that may be protonated or deprotonated (for example at physiological pH) the compound may be prepared and/or isolated as a pharmaceutically acceptable salt. It will be appreciated that the compound may be zwitterionic at a given pH. As used herein the expression “pharmaceutically acceptable salt” refers to the salt of a given compound, wherein the salt is suitable for administration as a pharmaceutical. For example, such salts may be formed by the reaction of an acid or a base with an amine or a carboxylic acid group respectively. Acid/base addition salts tend to be more soluble in aqueous solvents than the corresponding free acid/base forms.

In some embodiments, pharmaceutically acceptable salts of one or more compounds described herein are administered in a therapeutically effective amount to a subject. Such salts may be prepared by methods known in the art that involve reacting the compound with a suitable organic or inorganic acid or base. Representative organic salts include methanesulphonate, acetate, oxalate, adipate, alginate, aspartate, valerate, oleate, laurate, borate, benzoate, lactate, phosphate, toluenesulphonate (tosylate), citrate, malate, maleate, fumarate, succinate, tartrate, napsylate, methanesulphonate, 2-naphthalenesulphonate, nicotinate, benzenesulphonate, butyrate, camphorate, camphorsulphonate, cyclopentanepropionate, digluconate, dodecylsulphate, glucoheptanoate, glycerophosphate, heptanoate, hexanoate, undecanoate, 2-hydroxyethanesulphonate, ethanesulphonate, and the like. Representative inorganic salts can be formed from inorganic acids such as sulphate, bisulphate, hemisulphate, hydrochloride, chlorate, perchlorate, hydrobromide, hydroiodide, and the like. Examples of a base salt include ammonium salts; alkali metal salts such as sodium salts, potassium salts, and the like; alkaline earth metal salts such as calcium salts, magnesium salts, and the like; salts with organic bases such as dicyclohexylamine salts, N-methyl-D-glucamine, phenylethylamine, and the like; and salts with amino acids such as arginine, lysine, and the like. Such salts can readily be prepared employing methods well known in the art.

In some embodiments one or more compounds described herein is administered to a subject as a pro-drug. Pro-drugs are well known in the art and are compounds which are administered in a form that is then metabolized into a pharmacologically active drug. The term “pro-drug” is used in its broadest sense and encompasses those derivatives that are converted in vivo to the compounds of the invention. Such derivatives would readily occur to those skilled in the art and include, for example, compounds where a free hydroxy group is converted into an ester derivative or a ring nitrogen atom is converted to an N-oxide. Examples of ester derivatives include alkyl esters (for example acetates, lactates and glutamines), phosphate esters and those formed from amino acids (for example valine). Any compound that is a prodrug of a compound of the invention is within the scope and spirit of the invention. Conventional procedures for the preparation of suitable prodrugs according to the invention are described in text books, such as “Design of Prodrugs” Ed. H. Bundgaard, Elsevier, 1985—the entire contents of which is incorporated herein by reference.

The compounds of the invention may be in crystalline form or as solvates (e.g. hydrates) and it is intended that both forms are within the scope of the present invention. The term “solvate” is a complex of variable stoichiometry formed by a solute (in this invention, a compound of the invention) and a solvent. Such solvents should not interfere with the biological activity of the solute. Solvents may be, by way of example, water, ethanol or acetic acid. Methods of solvation are generally known within the art.

Unless otherwise stated, structures depicted herein are also meant to include all stereochemical forms of the structure; i.e., the R and S configurations for each asymmetric centre. Thus, single stereochemical isomers as well as enantiomeric and diastereomeric mixtures of the present compounds are within the scope of the invention. Thus, this invention encompasses each diastereomer or enantiomer substantially free of other isomers (>90%, and preferably >95%, free from other stereoisomers on a molar basis) as well as a mixture of such isomers.

Particular optical isomers can be obtained by resolution of the racemic mixtures according to conventional processes, e.g., by formation of diastereomeric salts, by treatment with an optically active acid or base. Examples of appropriate acids are tartaric, diacetyltartaric, dibenzoyltartaric, ditoluoyltartaric, and camphorsulphonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another method involves synthesis of covalent diastereomeric molecules by reacting compounds of the invention with an optically pure acid in an activated form or an optically pure isocyanate. The synthesized diastereomers can be separated by conventional means such as chromatography, distillation, crystallization or sublimation, and then hydrolysed to deliver the enantiomerically pure compound.

Optically active compounds of the invention can be obtained by using active starting materials. These isomers may be in the form of a free acid, a free base, an ester or a salt.

Where the compounds of the present invention require purification, chromatographic techniques such as high-performance liquid chromatography (HPLC) and reversed-phase HPLC may be used. The compounds may be characterised by mass spectrometry and/or other appropriate methods.

The present invention also provides a pharmaceutical composition comprising a therapeutically effective amount of a compound as hereinbefore defined, or a pharmaceutically acceptable salt thereof, together with at least one pharmaceutically acceptable carrier or diluent.

The term “composition” is intended to include the formulation of an active ingredient with encapsulating material as carrier, to give a capsule in which the active ingredient (with or without other carrier) is surrounded by carriers.

The compounds described herein are in certain embodiments administered individually, and in other embodiments are administered in combination, either with other compounds described herein, or with other therapeutic agents or both. The combination may allow for separate, sequential or simultaneous administration of the compound as hereinbefore described with the other active ingredient(s). The combination may be provided in the form of a pharmaceutical composition.

For example, in some embodiments two or more compounds described herein are administered to a subject together. When administering combinations of compounds to a subject, the dose of each individual compound may be less than the therapeutically effective amount such that the combined dose of the two or more compounds is equal to or greater than the therapeutically effective amount.

Furthermore, in some embodiments one or more compounds described herein is administered to a subject together with one or more additional agents. The additional agents may be, for example, agents that treat, inhibit and/or reverse amylin-amyloid fibril formation, islet of Langerhans Beta-cell death, the transition from soluble human amylin to insoluble human amylin and cytotoxic oligomer formation. In some embodiments the one or more additional agents may be compounds that prevent or treat amylin amyloid-associated diseases, such as type II diabetes.

For example, the one or more additional agents will in certain embodiments be selected from the group comprising blood glucose modulating agents, such as an insulin, an insulin analogue or derivative, Symlin, GLP-agonists, metformin, sulphonylureas, thiazolidinediones, SGLT2 inhibitors, and selective dipeptidyl peptidase (DPP-IV) inhibitors.

In various examples, the GLP-1 agonist is exenatide, liraglutide, lixisenatide, albiglutide, or dulaglutide, or any combination of two or more thereof.

In various examples, the selective dipeptidyl peptidase (DPP-IV) inhibitor is selected from the group comprising Sitagliptin, Vildagliptin, Saxagliptin, Linagliptin, Anagliptin, Teneligliptin, Alogliptin, Trelagliptin, Gemigliptin, Dutogliptin, and Omarigliptin.

In one example, the selective dipeptidyl peptidase (DPP-IV) inhibitor is selected from the group comprising alogliptin, linagliptin, saxagliptin, sitagliptin, Nesina, Tradjenta, Onglyza, and Januvia.

In some embodiments the one or more additional agents may for example be agents that reduce side effects associated with the compounds described herein.

In various embodiments one or more compounds described herein may be administered to a subject with one or more additional agents selected from the group comprising quinacrine, tetracycline and doxycycline.

In various embodiments employed in vitro, one or more compounds described herein may be administered to or contacted with a sample in vitro with one or more additional agents selected from the group comprising anthracene, phenanthrene, quinacrine, neutral red, chlorpromazine, acridine, acridine orange, methylene blue, phenodiazine, phenothiazine, tetracycline, doxycycline, Congo red, pyrene, chrysene, benz[a]anthracene, benz[m]anthracene, benzo[c]phenanthrene and tetracene.

When one or more compounds described herein are administered in combination with one or more additional agents, the dose of each compound administered may be less than the dose that would be administered if the compounds were administered separately.

In some embodiments when one or more compounds described herein are administered in combination with one or more additional agents, the dose of each compound administered may be greater than the dose that would be administered if the compounds were administered separately. For example, this may be the case if the one or more additional agents leads to reduced side effects, allowing a greater dose to be tolerated.

Structural elements such as groups, substituents, hetero ring members, numbers or other features, for example alkyl groups, groups like R1, R2, R3, etc., which can occur several times in the compounds of Formula I, Formula II, or Formula III, can all independently of one another have at each occurrence any of the indicated meanings and can in each case be identical to or different from one another. For example, the alkyl groups in a dialkylamino group can be identical or different.

As discussed above, the terms “including” and “comprising” are used herein in their open, non-limiting sense. As used herein, the terms “(C₁-C₆)” and so forth refer to moieties having 1 to 6 carbon atoms and so forth, respectively. Within composed terms like “hydroxy-(C₀-C₄)-alkyl” the option “(C₀)-alkyl refers to a bond (i.e. in this case a directly bound hydroxy group), or in case of an unsubstituted “(C₀)-alkyl” it refers to a hydrogen.

The term “alkyl”, as used herein, refers to saturated, monovalent hydrocarbon radicals. The term “alkenyl”, as used herein, refers to monovalent hydrocarbon radicals, which contain at least one carbon-carbon double bond, wherein each double bond can have E- or Z-configuration. The term “alkynyl”, as used herein, refers to monovalent hydrocarbon radicals, which contain at least one carbon-carbon triple bond. The alkyl, alkenyl and alkynyl groups can be linear, i.e. straight-chain, or branched. This also applies when they are part of other groups, for example alkyloxy groups (=alkoxy groups, O-alkyl groups), alkyloxycarbonyl groups or alkyl-substituted amino groups, or when they are substituted. Depending on the respective definition, the number of carbon atoms in an alkyl group can be 1-22, such as 1 to 12, such as 1, 2, 3, 4, 5 or 6, or 1, 2, 3, or 4. Examples of alkyl are methyl, ethyl, propyl including n-propyl and isopropyl, butyl including n-butyl, sec-butyl, isobutyl and tert-butyl, pentyl including n-pentyl, 1-methylbutyl, isopentyl, neopentyl and tert-pentyl, hexyl including n-hexyl, 3,3-dimethylbutyl and isohexyl. Double bonds and triple bonds in alkenyl groups and alkynyl groups respectively can be present in any positions. Examples of alkenyl and alkynyl are ethenyl, prop-1-enyl, prop-2-enyl (=allyl), but-2-enyl, 2-methylprop-2-enyl, 3-methylbut-2-enyl, hex-3-enyl, hex-4-enyl, prop-2-ynyl (=propargyl), but-2-ynyl, but-3-ynyl, hex-4-ynyl or hex-5-ynyl. Substituted alkyl groups, alkenyl groups and alkynyl groups can be substituted in any positions, provided that the respective compound is sufficiently stable and is suitable for the desired purpose such as use as a therapeutic substance. The prerequisite that a specific group and a compound of formula I, formula II, or formula III are sufficiently stable and suitable for the desired purpose such as use as a therapeutic substance, applies in general with respect to the definitions of all groups in the compounds of formula I, formula II, or formula III.

The term “alkenyloxy” as used herein accordingly represents an alkenyl-O— group, in which the alkenyl is a previously mentioned alkenyl group; for example, a 2-propenyloxy group, a 2-butenyloxy group, a 1-methyl-2-propenyloxy group, a 2-methyl-2-propenyloxy group and the like.

The term “alkanediyl” or “alkylene”, as used herein, refers to saturated, divalent hydrocarbon radicals. The term “alkenediyl”, as used herein, refers to divalent hydrocarbon radicals, which contain at least one carbon-carbon double bond, wherein each double bond can have E- or Z-configuration. The term “alkynediyl”, as used herein, refers to divalent hydrocarbon radicals, which contain at least one carbon-carbon triple bond. As far as applicable, the preceding explanations regarding alkyl, alkenyl and alkynyl groups apply correspondingly to alkanediyl, alkenediyl and alkynediyl groups, which thus can likewise be linear and branched. Examples of divalent alkyl groups are —CH₂— (=methylene), —CH₂—CH₂—, —CH₂—CH₂—CH₂—, —CH₂—CH₂—CH₂—CH₂—, —CH(CH₃)—, —C(CH₃)₂—, —CH(CH₃)—CH₂—, —CH₂—CH(CH₃)—, —C(CH₃)₂—CH₂— and —CH₂—C(CH₃)₂—.

The term “cycloalkyl”, as used herein, unless otherwise indicated, refers to a monovalent radical of a saturated hydrocarbon ring system, which is monocyclic. In a monocyclic cycloalkyl group the number of ring carbon atoms can be for example 3, 4, 5, 6, 7 or 8. In one embodiment of the invention, the number of ring carbon atoms in a cycloalkyl group, independently of the number of ring carbon atoms in any other cycloalkyl group is 3, 4, 5 or 6, in another embodiment 3 or 4, in another embodiment 3, in another embodiment 5 or 6, in another embodiment 5, in another embodiment 6. Examples of cycloalkyl groups are cyclopropyl, cyclobutyl, cyclopentyl and cyclohexyl.

The term “heterocycle”, as used herein, unless otherwise indicated, refers to a cycloalkyl as defined above, in which 1, 2, 3 or 4 carbon atoms are replaced by sulphur, nitrogen or oxygen atoms, provided that the heterocycloalkyl system is stable and suitable as a subgroup for the desired purpose of the compound of the formula I such as use as a drug substance. Depending on the definition of the respective heterocyclic group, in one embodiment of the invention the number of ring heteroatoms which can be present in a heterocyclic group, independently of the number of ring heteroatoms in any other heterocyclic group, is 1 or 2, in another embodiment 2, in another embodiment 1, wherein the ring heteroatoms can be identical or different. The heterocycloalkyl group can be attached by any ring carbon atom or saturated ring nitrogen or oxygen atom.

The term “alkyloxy” (also referred to as “alkoxy”), as used herein, unless otherwise indicated, refers to a radical —ORₐ where Rₐ is an alkyl as defined above, e.g., methoxy, ethoxy, propoxy, butoxy and the like.

The term “halo”, as used herein, unless otherwise indicated, refers to fluoro, chloro, bromo, or iodo, preferably fluoro and chloro. In some embodiments halo is F. It will be understood that in certain circumstances a fluorine atom may function as an isostere for hydrogen, and accordingly the skilled person may in certain embodiments substitute one or more hydrogen atoms in an alkyl, alkenyl, aryl and/or cycloalkyl group, for example, for fluorine atom(s).

The term “haloalkyl”, as used herein, unless otherwise indicated, refers to alkyl substituted with one or more, preferably one, two or three, same or different halo atoms, e.g., —CH₂Cl, —CF₃, —CH₂CF₃, —CH₂CCl₃, and the like.

The term “haloalkoxy”, as used herein, unless otherwise indicated, refers to a radical —OR_{b} where R_{b} is an haloalkyl as defined above, e.g., trifluoromethoxy, trichloroethoxy, 2,2-dichloropropoxy, and the like.

The term “acyl”, as used herein, unless otherwise indicated, refers to a radical —C(O)R_{c} where R_{c} is hydrogen, alkyl, or haloalkyl as defined herein, e.g., formyl, acetyl, trifluoroacetyl, butanoyl, and the like.

The term “aryl”, as used herein, unless otherwise indicated, refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The aryl group may be substituted or unsubstituted. Unless specifically stated otherwise, “substituted aryl” refers to the aryl group being substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, phenoxy, heteroaryl, heteroaryloxy, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

As used herein, the prefix “hetero”, as a part of a group, refers to the presence of at least one heteroatom in at least one carbon atom-containing ring of that group. Each ring of the hetero-containing group may have 1, 2, 3 or 4 heteroatoms selected from N, O and/or S, where the N and S heteroatoms may optionally be oxidized and the N heteroatoms may optionally be quaternized. Preferably the heteroatom will be N or O.

The term “heteroaryl”, as used herein, unless otherwise indicated, refers to a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system. Examples of typical heteroaryl rings include 5-membered monocyclic ring groups such as thienyl, pyrrolyl, imidazolyl, pyrazolyl, furyl, isothiazolyl, furazanyl, isoxazolyl, thiazolyl and the like; 6-membered monocyclic groups such as pyridyl, pyrazinyl, pyrimidinyl, pyridazinyl, triazinyl and the like; and polycyclic heterocyclic ring groups such as benzo[b]thienyl, naphtho[2,3-b]thienyl, thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxalinyl, quinazolinyl, benzothiazole, benzimidazole, tetrahydroquinoline cinnolinyl, pteridinyl, carbazolyl, beta-carbolinyl, phenanthridinyl, acridinyl, perimidinyl, phenanthrolinyl, phenazinyl, isothiazolyl, phenothiazinyl, phenoxazinyl, and the like (see e.g. Katritzky, Handbook of Heterocyclic Chemistry). Further specific examples of heteroaryl rings include 2-furanyl, 3-furanyl, N-imidazolyl, 2-imidazolyl, 4-imidazolyl, 5-imidazolyl, 3-isoxazolyl, 4 isoxazolyl, 5 isoxazolyl, 2-oxadiazolyl, 5-oxadiazolyl, 2-oxazolyl, 4-oxazolyl, oxazolyl, 1-pyrrolyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indolyl, isoindolyl, acridinyl, or benzoisoxazolyl. Heteroaryl groups further include a group in which a heteroaromatic ring is fused to one or more aromatic or nonaromatic rings where the radical or point of attachment is on the heteroaromatic ring. Examples include tetrahydroquinoline, tetrahydroisoquinoline, and pyrido[3,4-d]pyrimidinyl, imidazo[1,2-a]pyrimidyl, imidazo[1,2-a]pyrazinyl, imidazo[1,2-a]pyridinyl, imidazo[1,2-c]pyrimidyl, pyrazolo[1,5-a][1,3,5]triazinyl, pyrazolo[1,5c]pyrimidyl, imidazo[1,2-b]pyridazinyl, imidazo[1,5-a]pyrimidyl, pyrazolo[1,5-b][1,2,4]triazine, quinolyl, isoquinolyl, quinoxalyl, imidazotriazinyl, pyrrolo[2,3-d]pyrimidyl, triazolopyrimidyl, pyridopyrazinyl. Unless specifically stated otherwise, “substituted heteroaryl” refers to the heteroaryl group being substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, haloalkyl, haloalkoxy, carboxy, alkoxycarbonyl, amino, alkylamino, dialkylamino, aryl, heteroaryl, carbocycle or heterocycle (wherein the aryl, heteroaryl, carbocycle or heterocycle may be optionally substituted).

The term “carbocycle” as used herein, unless otherwise indicated, refers to a saturated, unsaturated or aromatic mono- or polycyclic ring system having 3 to 14 ring carbon atoms. The term “carbocycle”, whether saturated or partially unsaturated, also refers to rings that are optionally substituted. The term “carbocycle” includes aryl. The term “carbocycle” also includes aliphatic rings that are fused to one or more aromatic or nonaromatic rings, such as in a decahydronaphthyl or tetrahydronaphthyl, where the radical or point of attachment is on the aliphatic ring. The carbocycle group may be substituted or unsubstituted. Unless specifically stated otherwise, “substituted carbocycle” refers to the carbocycle group being substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

The term “heterocycle” refers to a saturated, unsaturated or aromatic cyclic ring system having 3 to 14 ring atoms in which one, two or three ring atoms are heteroatoms selected from N, O, or S(O)ₘ (where m is an integer from 0 to 2), the remaining ring atoms being C, where one or two C atoms may optionally be replaced by a carbonyl group. The term “heterocycle” includes heteroaryl. Unless specifically stated otherwise, “substituted heterocyclyl” refers to the heterocyclyl ring being substituted independently with one or more, preferably one, two, or three substituents selected from alkyl (wherein the alkyl may be optionally substituted with one or two substituents), haloalkyl, cycloalkylamino, cycloalkylalkyl, cycloalkylaminoalkyl, cycloalkylalkylaminoalkyl, cyanoalkyl, halo, nitro, cyano, hydroxy, alkoxy, amino, alkylamino, dialkylamino, hydroxyalkyl, carboxyalkyl, aminoalkyl, alkylaminoalkyl, dialkylaminoalkyl, aralkyl, heteroaralkyl, aryl, heteroaryl, carbocycle, heterocycle (wherein the aryl, heteroaryl, carbocycle or heterocycle may be optionally substituted), aralkyl, heteroaralkyl, saturated or unsaturated heterocycloamino, saturated or unsaturated heterocycloaminoalkyl, and —COR_{d} (where R_{d} is alkyl). More specifically the term heterocyclyl includes, but is not limited to, tetrahydropyranyl, 2,2-dimethyl-1,3-dioxolane, piperidino, N-methylpiperidin-3-yl, piperazino, N-methylpyrrolidin-3-yl, pyrrolidino, morpholino, 4-cyclopropylmethylpiperazino, thiomorpholino, thiomorpholino-1-oxide, thiomorpholino-1,1-dioxide, 4-ethyloxycarbonylpiperazino, 3-oxopiperazino, 2-imidazolidone, 2-pyrrolidinone, 2-oxohomopiperazino, tetrahydropyrimidin-2-one, and the derivatives thereof, including 2-methyl-4,5,6,7-tetrahydro-1H-pyrrolo[2,3-c]pyridinyl. In certain embodiments, the heterocycle group is optionally substituted with one or two substituents independently selected from halo, alkyl, alkyl substituted with carboxy, ester, hydroxy, alkylamino, saturated or unsaturated heterocycloamino, saturated or unsaturated heterocycloaminoalkyl, or dialkylamino.

As used herein “carboxy” refers to the group —CO₂H. Likewise, “alkylcarboxy” refers to the group —CO₂(alkyl), whereas “carboxyalkyl” refers to the group -alkyl-CO₂H

As used herein, “optional” or “optionally” means that the subsequently described event or circumstance may but need not occur, and that the description includes instances where the event or circumstance occurs and instances in which it does not. For example, “heterocyclic group optionally substituted with an alkyl group” means that the alkyl may but need not be present, and the description includes situations where the heterocycle group is substituted with an alkyl group and situations where the heterocycle group is not substituted with the alkyl group.

Unless specifically stated otherwise, the term “substituted” as used herein means any of the above groups (e.g., alkyl, aryl, heteroaryl, carbocycle, heterocycle, etc.) wherein at least one hydrogen atom is replaced with a substituent. “Substituents” as contemplated herein, if not specified, include halogen, hydroxy, oxo (═O), thio (═S), cyano, nitro, amino, aminoalkyl, alkylamino, dialkylamino, alkyl, cycloalkyl, alkenyl, alkyloxy, thioalkyl, haloalkyl (e.g., —CF₃), hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocycloalkyl, substituted heterocycloalkyl, —NRₑR_{f}, —NRₑC(═O)R_{f}, —NRₑC(═O)NRₑR_{f}, —NRₑC(═O)OR_{f}, —NRₑSO₂R_{f}, —ORₑ, —C(═O)Rₑ, —C(═O)ORₑ, —OC(═O)Rₑ, —C(═O)NRₑR_{f}, —OC(═O)NRₑR_{f}, —SH, —SRₑ, —SORₑ, —S(═O)₂Rₑ, —OS(═O)₂Rₑ, —S(═O)₂₀Rₑ, wherein Rₑ and R_{f} are the same or different and independently hydrogen, alkyl, haloalkyl, substituted alkyl, cycloalkyl, substituted cycloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocycloalkyl or substituted heterocycloalkyl.

In various embodiments, substituents are selected from halo, C₁₋₂₂alkyl, C₂₋₂₂alkenyl, C₆₋₁₆aryl, C₅₋₁₄heteroaryl, C₃₋₁₂cycloalkyl, C₃₋₁₂heterocycloalkyl, hydroxy, hydroxyC₁₋₂₂alkyl, amino, aminoC₁₋₂₂alkyl, C₁₋₂₂alkyloxy, C₁₋₂₂alkylamino, (C₁₋₂₂alkyl)(C₁₋₂₂alkyl)amino, C₁₋₂₂alkylcarbonyloxy, C₁₋₂₂alkyloxycarbonyl, C₂₋₂₂alkenylcarbonyloxy, C₂₋₂₂alkenyloxycarbonyl, C₆₋₁₆arylcarbonyloxy, C₆₋₁₆aryloxycarbonyl, C₃₋₁₂cycloalkylcarbonyloxy, C₃₋₁₂cycloalkyloxycarbonyl, C₁₋₂₂alkylcarbonylamino, C₂₋₂₂alkenylcarbonylamino, C₆-16arylacylamino, C₃₋₁₂cycloalkylcarbonylamino, C₁₋₂₂alkylaminocarbonyl, C₂₋₂₂alkenylaminocarbonyl, C₆₋₁₆arylaminoacyl, C₃₋₁₂cycloalkylaminocarbonyl, carboxy, carboxyalkyl, carboxyalkenyl, ═O, ═S, ═NH, ═NNR₃₂R₃₃, ═NNHC(O)R₃₂, ═NNHCO₂R₃₂, and ═NNHSO₂R₃₂, wherein R₃₂ and R₃₃ at each occurrence are independently hydrogen, optionally substituted alkyl, optionally substituted alkenyl, optionally substituted alkynyl, optionally substituted cycloalkyl, optionally substituted cycloalkenyl, optionally substituted cycloalkynyl, optionally substituted aryl, optionally substituted heteroaryl, and optionally substituted heterocycloalkyl.

### EXAMPLES

It will be appreciated by those skilled in the art that the synthetic methods described below are readily adapted to prepare compounds of Formula I or Formula II as applicable.

### Example 1: Synthesis of Compound (1)

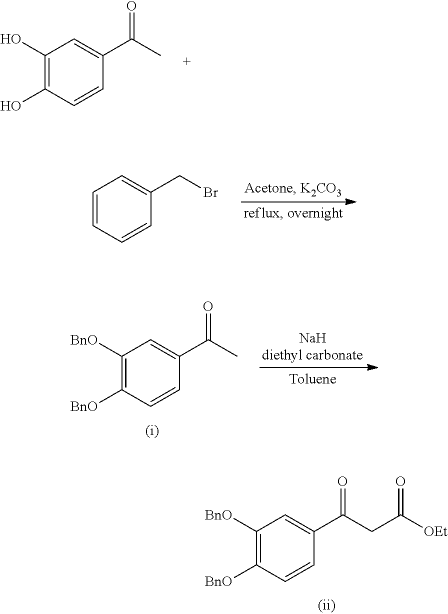 [SMILES: CC(=O)c1ccc(O)c(O)c1 ; BrCc1ccccc1 ; CC(=O)c1ccc(OCc2ccccc2)c(OCc2ccccc2)c1 ; CCOC(=O)CC(=O)c1ccc(OCc2ccccc2)c(OCc2ccccc2)c1]

K₂CO₃ (2 eq.) was added to a solution of dihydroxyacetophenone in acetone and the mixture stirred for 30 min at r.t. Benzyl bromide (2.2 eq.) was added and the mixture refluxed overnight. The mixture was concentrated in vacuo, and suspended in ethyl acetate. The suspension was washed with water and brine. The organic layer was dried over Na₂SO₄ and concentrated under reduced pressure. The crude product was then purified by column chromatography to give compound (i).

Diethylcarbonate (2.5 eq.) was added to a suspension of sodium hydride (60%, 2.5 eq.) in anhydrous toluene and the mixture was heated to reflux. A solution of compound (i) in toluene was added dropwise and the resulting mixture was refluxed for further 30 min until the production of hydrogen ceased. After cooling, a mixture of acetic acid and water (1:1) was added. Ice water (20 mL) was then added. The organic layer was separated and the water layer extracted with ethyl acetate. The organic layer was combined and washed with brine several times, dried over Na₂SO₄ and concentrated under reduced pressure. The residue was dried to yield compound (ii) which was used directly in the next step.

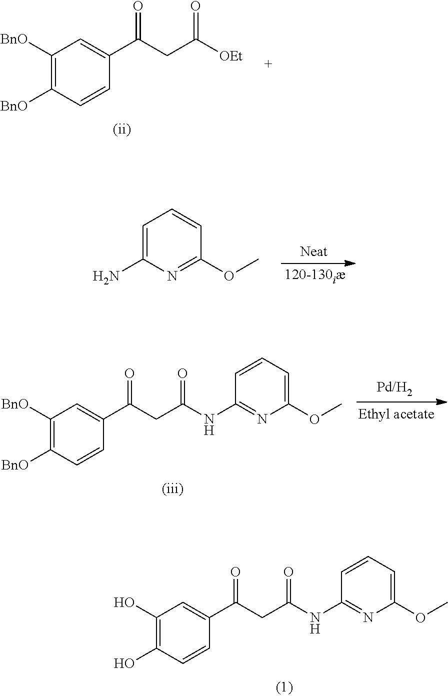

A mixture of compound (ii) and 2-amino-6-methoxypyridine was stirred at 120° C. for 6-8 hours without any solvent. After cooling, ethyl acetate was added and the organic layer was concentrated under reduced pressure. The crude product was then purified by column chromatography to give compound (iii).

Pd/C was added to a solution of compound (iii) in ethyl acetate, and the mixture stirred at r.t for 2.5 hours under an atmosphere of hydrogen. After completion, the Pd/C was filtered with DCM/MeOD, and the solvent was concentrated under reduced pressure and to yield compound (1). ¹H NMR (300 MHz, DMSO) δ 10.44 (s, 1H), 9.98 (s, 1H), 9.42 (s, 1H), 7.67 (d, J=5.6 Hz, 2H), 7.38 (dt, J=6.6, 2.1 Hz, 2H), 6.84 (d, J=8.1 Hz, 1H), 6.56-6.49 (m, 1H), 4.09 (s, 2H), 3.84 (d, J=7.6 Hz, 3H). LC-MS (ESI) calculated for [M+H]⁺ 303.1; Found 303.1.

### Example 2: Synthesis of Compound (2)

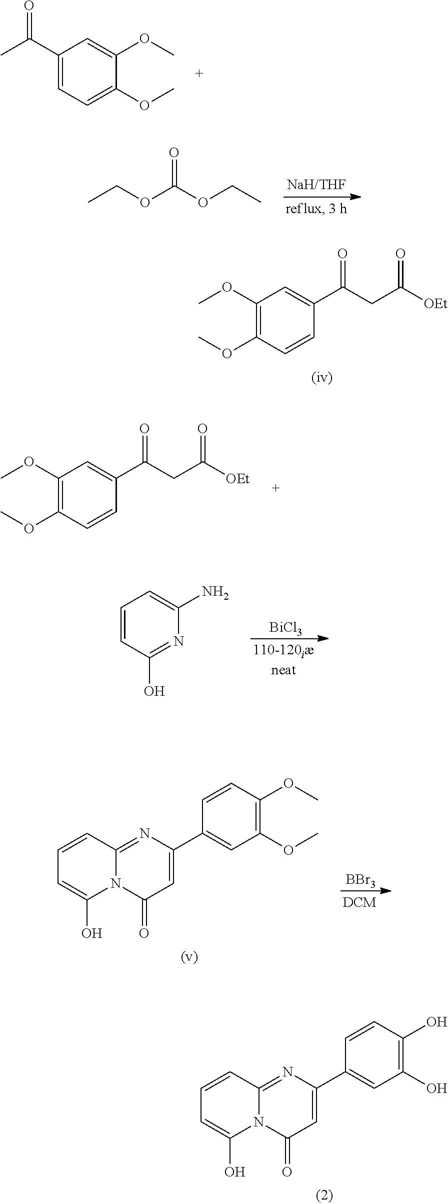

Diethylcarbonate (2.5 eq.) was added to a suspension of sodium hydride (60%, 2.5 eq.) in anhydrous toluene and the mixture was heated to reflux. A solution of 3,4-dimethoxyacetophenone in toluene was added dropwise and the resulting mixture was refluxed for further 30 min until the production of hydrogen ceased. After cooling, a mixture of acetic acid and water (1:1) was added. Ice water (20 mL) was added, the organic layer was separated and the water layer was extracted with ethyl acetate. The organic layer was combined and washed with brine several times, dried over Na₂SO₄ and concentrated under reduced pressure. The residue was dried to yield compound (xii) which was used directly in the next step.

BiCl₃ (10 mol %) was added to a mixture of compound (xii) and 6-aminopyridin-2-ol and the mixture was stirred at 120° C. for 6-8 hours. After cooling, ethyl acetate was added and the organic layer was concentrated under reduced pressure. The crude product was then purified by column chromatography to give compound (xiii).

BBr₃ was added dropwise at 0° C. to a solution of compound (Xiii) in anhydrous DCM. After the reaction was complete, water was added to quench the reaction and the mixture filtered. The filter cake was collected and then purified by column chromatography to yield compound (5). ¹H NMR (400 MHz, DMSO) δ 8.25 (s, 1H), 7.62 (d, J=8.6 Hz, 1H), 7.13 (s, 2H), 6.87 (dd, J=8.5, 5.1 Hz, 2H), 6.78 (dd, J=8.1, 2.0 Hz, 1H), 6.44 (d, J=8.7 Hz, 1H), 5.89 (s, 1H); ¹³C NMR (101 MHz, DMSO) δ 161.16 (d, J=5.3 Hz), 147.66 (s), 146.00 (s), 137.37 (s), 126.36 (s), 120.30 (s), 116.27 (d, J=19.0 Hz), 106.87 (s), 106.63 (s), 102.64 (s), 40.60 (s), 40.39 (s), 40.18 (s), 39.97 (s), 39.76 (s), 39.55 (s), 39.35 (s); LC-MS (ESI) calculated for [M+H]+ 271.1; Found 271.1.

### Example 3: Synthesis of Compound (3)

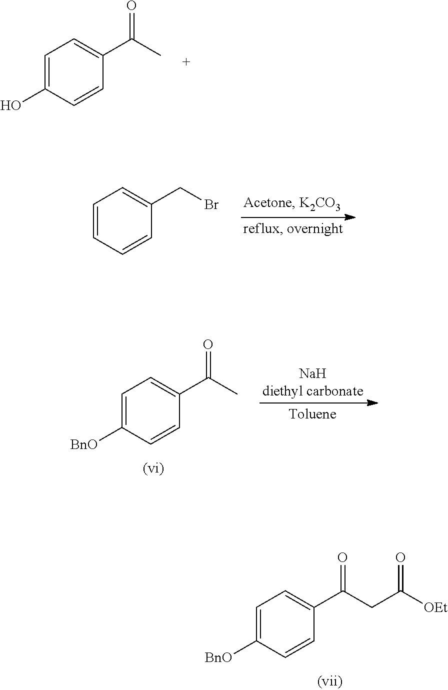

K₂CO₃ (1 eq.) was added to a solution of 4-hydroxyacetophenone in acetone and the mixture stirred for 30 min at r.t. Benzyl bromide (1.1 eq.) was added and the mixture refluxed overnight. The mixture was then concentrated in vacuo and suspended in ethyl acetate. The suspension was washed with water and brine. The organic layer was dried over Na₂SO₄ and concentrated under reduced pressure. The crude product was then purified by column chromatography to give compound (vi).

Diethylcarbonate (2.5 eq.) was added to a suspension of sodium hydride (60%, 2.5 eq.) in anhydrous toluene and the mixture was heated to reflux. A solution of compound (vi) in toluene was then added dropwise and the resulting mixture refluxed for further 30 min until the production of hydrogen ceased. After cooling, a mixture of acetic acid and water (1:1) was added. Ice water (20 mL) was added, the organic layer separated and the water layer extracted with ethyl acetate. The organic layer was washed with brine several times, dried over Na₂SO₄ and concentrated under reduced pressure. The residue was dried to yield compound (vii) which was used directly in the next step.

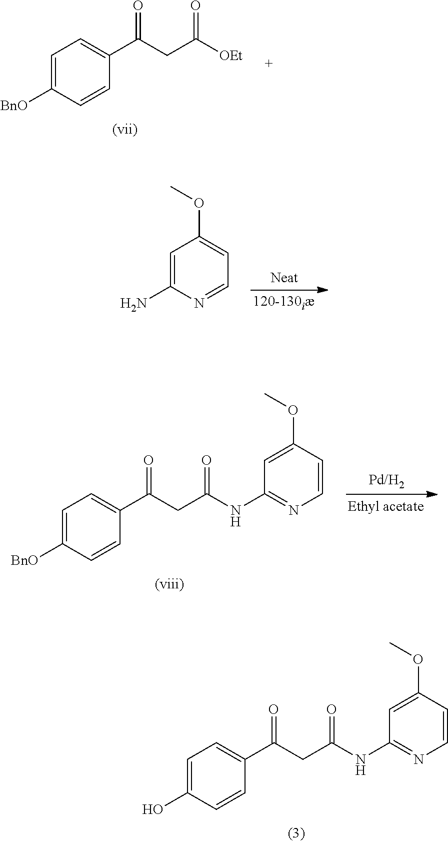

A mixture of compound (vii) and 2-amino-6-methoxypyridine was stirred at 120° C. for 6-8 hours without any solvent. After cooling, ethyl acetate was added and the organic layer was concentrated under reduced pressure. The crude product was then purified by column chromatography to give compound (viii).

Pd/C was added to a solution of compound (viii) in ethyl acetate and the mixture stirred at r.t for 2.5 hours under an atmosphere of hydrogen. After completion, the Pd/C was filtered with DCM/MeOD, and the solvent was concentrated under reduced pressure and to yield compound (3). ¹H NMR (400 MHz, DMSO) δ 11.88 (s, 1H), 10.55 (s, 1H), 8.26 (dd, J=6.8, 4.0 Hz, 1H), 7.90 (d, J=8.7 Hz, 2H), 7.68 (d, J=8.8 Hz, 1H), 7.21-7.01 (m, 2H), 7.04-6.74 (m, 2H), 6.10 (d, J=27.7 Hz, 1H), 4.29 (s, 2H), 3.99 (d, J=5.0 Hz, 3H); ¹³C NMR (101 MHz, DMSO) δ 192.15 (s), 170.93 (s), 169.31 (s), 163.22 (s), 161.82 (s), 149.99 (s), 142.61 (s), 131.59 (s), 128.48 (s), 127.87 (s), 123.43 (s), 116.31 (s), 115.88 (s), 114.56 (s), 109.32 (s), 98.86 (s), 87.02 (s), 57.49 (s), 48.11 (s); LC-MS (ESI) calculated for [M+H]+ 287.1; Found 287.1.

### Example 4: Synthesis of Compound (4)

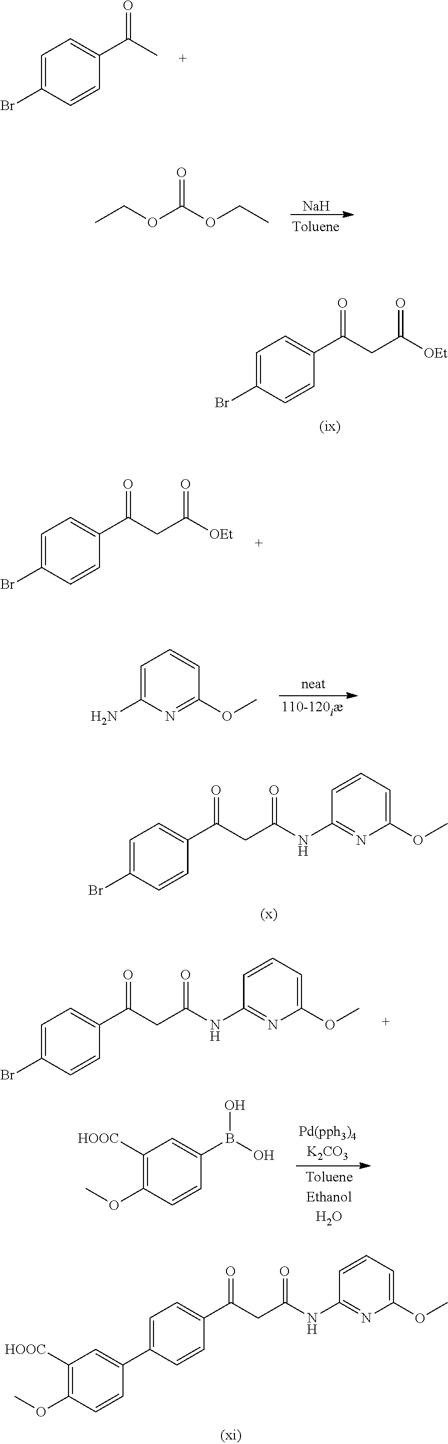

Diethylcarbonate (2.5 eq.) was added to a suspension of sodium hydride (60%, 2.5 eq.) in anhydrous toluene and the mixture was heated to reflux. A solution of 4′-bromoacetophenone in toluene was added dropwise and the resulting mixture was refluxed for a further 30 min until the production of hydrogen ceased. After cooling, a mixture of acetic acid and water (1:1) was added. Ice water (20 mL) was added. The organic layer was separated and the water layer was extracted with ethyl acetate. The organic layer was combined and washed with brine several times, dried over Na₂SO₄ and concentrated under reduced pressure. The residue was dried to yield compound (ix) which was used directly in the next step.

A mixture of compound (ix) and 2-amino-6-methoxypyridine was stirred at 120° C. for 6-8 hours without any solvent. After cooling, ethyl acetate was added and the organic layer was concentrated under reduced pressure. The crude product was then purified by column chromatography to give compound (x).

A tube was charged with Pd(pph₃)₄ in toluene:ethanol:H₂O (3:2:1), followed by the addition of compound (x), K₂CO₃ and 3-carboxy-4-methoxybenzeneboronic. The reaction was carried out at 80° C. for 6 h under nitrogen. The mixture was diluted with DCM and washed with brine for three times. The organic layer was dried with Na₂SO₄. The crude product was then purified by column chromatography to give compound (xi).

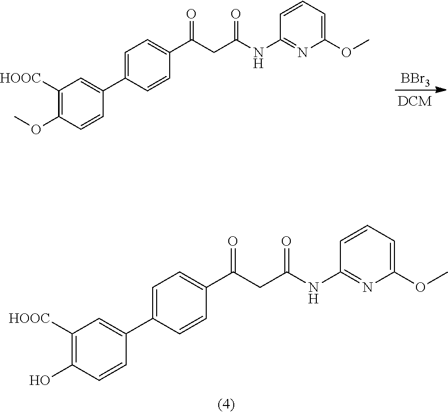

BBr₃ was added dropwise at 0° C. to a solution of compound (xi) in anhydrous DCM. After the reaction was complete, water was added to quench the reaction and the mixture filtered. The filter cake was collected and then purified by column chromatography to yield compound (4). ¹H NMR (400 MHz, DMSO) δ 10.53 (s, 1H), 8.14 (dd, J=4.3, 2.5 Hz, 1H), 8.06 (d, J=8.4 Hz, 2H), 7.95 (dt, J=8.3, 4.1 Hz, 1H), 7.89-7.79 (m, 3H), 7.70 (dd, J=11.7, 4.9 Hz, 2H), 7.10 (dd, J=8.7, 3.1 Hz, 1H), 6.61-6.47 (m, 1H), 6.36 (s, 1H), 4.28 (s, 2H), 3.85 (s, 3H); LC-MS (ESI) calculated for [M+H]+ 407.1; Found 407.1.

### Example 5: Synthesis of Compound (5)

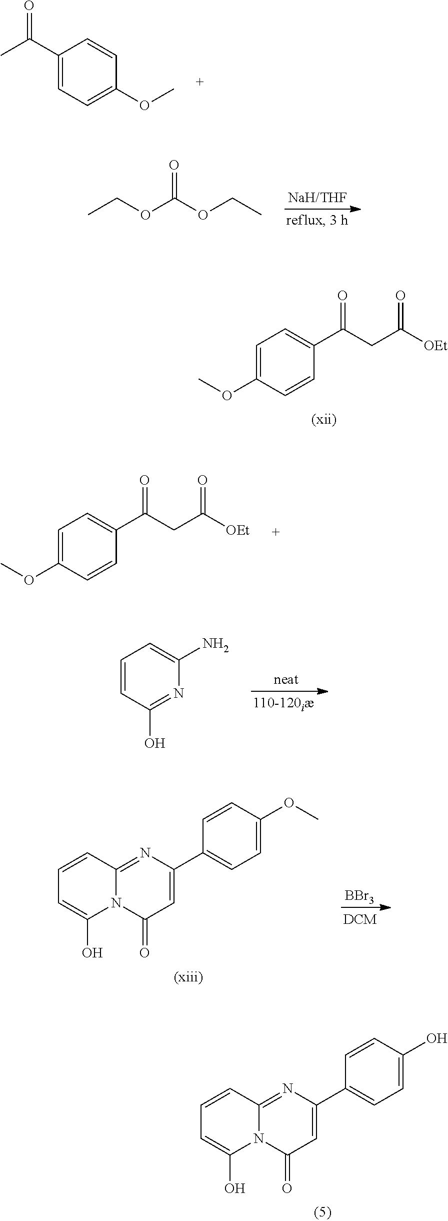

Diethylcarbonate (2.5 eq.) was added to a suspension of sodium hydride (60%, 2.5 eq.) in anhydrous toluene and the mixture was heated to reflux. A solution of 4-hydroxyacetophenone in toluene was added dropwise and the resulting mixture was refluxed for a further 30 min until the production of hydrogen ceased. After cooling, a mixture of acetic acid and water (1:1) was added. Ice water (20 mL) was added, the organic layer was separated and the water layer was extracted with ethyl acetate. The organic layer was combined and washed with brine several times, dried over Na₂SO₄ and concentrated under reduced pressure. The residue was dried to yield compound (xii) which was used directly in the next step.

BiCl₃ (10 mol %) was added to a mixture of compound (xii) and 6-aminopyridin-2-ol and the mixture was stirred at 120° C. for 6-8 hours. After cooling, ethyl acetate was added and the organic layer was concentrated under reduced pressure. The crude product was then purified by column chromatography to give compound (xiii).

BBr₃ was added dropwise at 0° C. to a solution of compound (xiii) in anhydrous DCM. After the reaction was complete, water was added to quench the reaction and the mixture filtered. The filter cake was collected and then purified by column chromatography to yield compound (5). ¹H NMR (300 MHz, DMSO) δ 10.44 (s, 1H), 9.95 (s, 1H), 9.38 (s, 1H), 8.01 (d, J=5.7 Hz, 1H), 7.36 (dt, J=4.9, 2.1 Hz, 2H), 7.09-6.93 (m, 2H), 6.82 (d, J=8.2 Hz, 1H), 4.01 (s, 2H), 3.80 (d, J=3.4 Hz, 4H); ¹³C NMR (101 MHz, DMSO) δ 161.17 (d, J=9.1 Hz), 160.37 (s), 159.35 (s), 156.08 (s), 137.27 (s), 130.43 (s), 125.97 (s), 116.09 (s), 107.09 (s), 106.70 (s), 102.63 (s), 40.59 (s), 40.38 (s), 40.18 (s), 39.97 (s), 39.76 (s), 39.55 (s), 39.34 (s); LC-MS (ESI) calculated for [M+H]+ 255.1; Found 255.1.

### Example 6: Synthesis of Compound (6)

Compound (7) was prepared according to the methodology described in Example 2 to give a gold-coloured solid. ¹H NMR (400 MHz, DMSO) δ 9.79 (s, 1H), 7.50 (d, J=8.6 Hz, 1H), 7.34 (t, J=7.8 Hz, 1H), 7.18 (s, 2H), 6.95-6.72 (m, 3H), 6.44 (d, J=8.6 Hz, 1H), 5.96 (s, 1H); ¹³C NMR (101 MHz, DMSO) δ 161.33 (s), 161.02 (s), 160.30 (s), 158.02 (s), 156.19 (s), 137.11 (s), 136.75 (s), 130.49 (s), 119.32 (s), 116.97 (s), 115.41 (s), 107.73 (s), 106.80 (s), 102.54 (s), 40.60 (s), 40.39 (s), 40.18 (s), 39.97 (s), 39.76 (s), 39.55 (s), 39.34 (s); LC-MS (ESI) calculated for [M+H]+ 255.1; Found 255.1.

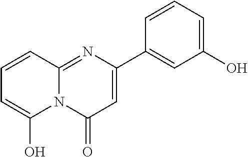 [SMILES: O=c1cc(-c2cccc(O)c2)nc2cccc(O)n12]

### Example 7: Synthesis of Compound (7)

Compound (8) was prepared according to the methodology described in Example 2 to give a yellow solid. ¹H NMR (400 MHz, DMSO) δ 7.62 (d, J=8.7 Hz, 1H), 7.14 (d, J=13.0 Hz, 2H), 7.10 (s, 1H), 7.08 (d, J=1.9 Hz, 1H), 7.05 (dd, J=8.2, 2.0 Hz, 1H), 6.44 (d, J=8.7 Hz, 1H), 6.03 (s, 1H), 3.83 (d, J=6.1 Hz, 6H); ¹³C NMR (101 MHz, DMSO) δ 161.17 (d, J=13.0 Hz), 160.36 (s), 155.95 (s), 150.38 (s), 149.26 (s), 137.32 (s), 127.78 (s), 121.51 (s), 112.40 (s), 112.18 (s), 107.55 (s), 106.79 (s), 102.64 (s), 56.08 (d, J=1.7 Hz), 40.49 (d, J=21.0 Hz), 40.18 (s), 40.12-40.08 (m), 39.97 (s), 39.76 (s); LC-MS (ESI) calculated for [M+H]+ 299.1; Found 299.1.

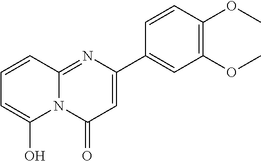 [SMILES: COc1ccc(-c2cc(=O)n3c(O)cccc3n2)cc1OC]

### Example 8: Synthesis of Compound (8)

Compound (9) was prepared according to the methodology described in Example 2 to give a yellow solid. ¹H NMR (400 MHz, DMSO) δ 12.86 (s, 1H), 10.53 (s, 1H), 8.06 (d, J=8.0 Hz, 1H), 8.00 (d, J=2.2 Hz, 1H), 7.93 (d, J=6.9 Hz, 1H), 7.85 (d, J=8.4 Hz, 2H), 7.69 (d, J=4.3 Hz, 2H), 7.26 (d, J=8.8 Hz, 1H), 6.67-6.43 (m, 1H), 5.76 (s, 1H), 4.28 (s, 1H), 3.87 (d, J=14.9 Hz, 6H); ¹³C NMR (101 MHz, DMSO) δ 167.74 (s), 162.88 (s), 158.69 (s), 150.19 (s), 144.12 (s), 141.48 (s), 135.16 (s), 131.70 (s), 130.87 (s), 129.44 (d, J=25.2 Hz), 126.85 (s), 122.81 (s), 113.61 (s), 105.74 (s), 56.44 (s), 53.59 (s), 40.59 (s), 40.39 (s), 40.18 (s), 39.97 (s), 39.76 (s), 39.57 (s); LC-MS (ESI) calculated for [M+H]+ 421.1; Found 421.1.

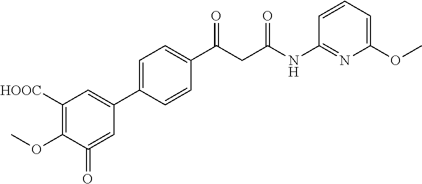

### Example 9: Synthesis of Compound (9)

Compound (10) was prepared according to the methodology described in Example 2 to give a yellow solid. ¹H NMR (400 MHz, DMSO) δ 10.63 (s, 1H), 9.97 (s, 1H), 9.40 (s, 1H), 8.14 (t, J=5.8 Hz, 1H), 7.72 (s, 1H), 7.50-7.23 (m, 2H), 6.84 (dd, J=8.2, 3.2 Hz, 1H), 6.72 (dd, J=5.8, 2.3 Hz, 1H), 4.08 (s, 2H), 3.83 (d, J=11.5 Hz, 3H); ¹³C NMR (101 MHz, DMSO) δ 193.04 (s), 167.19 (d, J=15.5 Hz), 163.51 (s), 153.85 (s), 151.56 (s), 149.45 (s), 145.74 (s), 128.71 (s), 122.35 (s), 115.53 (d, J=6.2 Hz), 106.87 (s), 99.01 (s), 55.75 (s), 47.90 (s), 40.60 (s), 40.39 (s), 40.18 (s), 39.97 (s), 39.76 (s), 39.55 (s), 39.34 (s); LC-MS (ESI) calculated for [M+H]+ 303.1; Found 303.1.

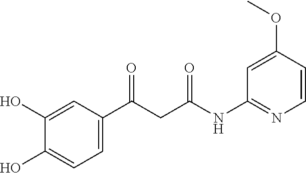 [SMILES: COc1ccnc(NC(=O)CC(=O)c2ccc(O)c(O)c2)c1]

### Example 10: Synthesis of Compound (10)

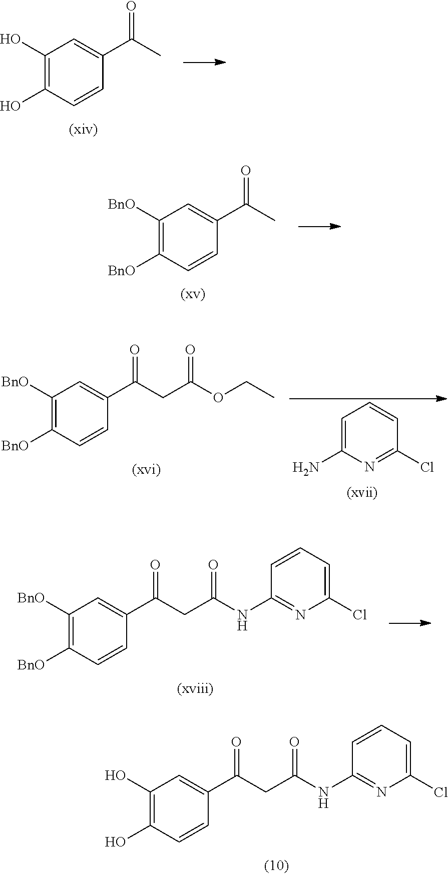 [SMILES: CC(=O)c1ccc(O)c(O)c1 ; CC(=O)c1ccc(OCc2ccccc2)c(OCc2ccccc2)c1 ; CCOC(=O)CC(=O)c1ccc(OCc2ccccc2)c(OCc2ccccc2)c1 ; Nc1cccc(Cl)n1 ; O=C(CC(=O)c1ccc(OCc2ccccc2)c(OCc2ccccc2)c1)Nc1cccc(Cl)n1 ; O=C(CC(=O)c1ccc(O)c(O)c1)Nc1cccc(Cl)n1]

To a solution of compound (xiv) (15.2 g, 100 mmol) in acetone (100 mL) was added K₂CO₃ (41.4 g, 300 mmol), and the mixture was stirred for 30 min at room temperature. Benzyl bromide (30 mL, 250 mmol) was added, and the mixture was refluxed overnight. The solvent was removed, and the residue was suspended in diethyl ether (200 mL). The suspension was washed with water (2×100 mL) and brine (2×100 mL) and dried over Na₂SO₄. Removal of the solvent under reduced pressure gave white crystals that were suspended in petroleum ether (300 mL) and filtered to yield compound (xv).

NaH (4.1 g, ca. 60% in mineral oil) was washed with twice with a small amount of hexanes in a two-neck round-bottom flask equipped with a reflux condenser and a magnetic stirring bar. The washed NaH thus obtained was suspended in dry THF (200 mL) and cooled to 0° C. Diethyl carbonate (9.5 mL, 76.3 mmol) and the corresponding ketone (xv) (17.0 g, 50.9 mmol) were added successively. After 10 minutes the reaction mixture was allowed to warm to ambient temperature. Once the reaction mixture had reached ambient temperature it was slowly heated to reflux in an oil bath. After full conversion of the ketone the reaction mixture was cooled to 0° C. in an ice bath. The reaction was quenched with 1M aq. NaHSO₄ and diluted with Et₂O. The layers were separated and the aqueous layer was extracted with Et₂O three times. The combined organic layers were washed successively with sat. aq. NaHCO₃ and brine, dried over Na₂SO₄, filtered and concentrated to obtain compound (xvi).

A solution of compound (xvi) (10.0 g, 24.7 mmol) was heated to 130° C. To the boiling compound (xvi) was added compound (xvii) (3.2 g, 24.7 mmol) in portions during 1 h, and the liberated EtOH was removed by distillation. Cooling the mixture at 0° C. afforded the product, which was collected by filtration under suction.

To a solution of O-benzyl-protected compound (xviii) (14.6 g, 30 mmol) in a mixture of absolute ethanol (200 mL) and THF (200 mL) was added 10% Pd/C (1.5 g), and the reaction mixture was stirred under a hydrogen atmosphere for 10 h. The catalyst was filtered off, and the solvent was removed under reduced pressure. The crude product was purified by column chromatography with dichloromethane/methanol as the eluent to afford compound (10). ¹H NMR (400 MHz, DMSO) δ 14.58-14.53 (m, 1H), 10.97 (s, 1H), 9.69 (d, 3=204.4 Hz, 1H), 8.06 (d, J=8.2 Hz, 1H), 7.84 (t, J=8.0 Hz, 1H), 7.37 (dd, J=11.0, 2.8 Hz, 1H), 7.21 (d, J=7.7 Hz, 1H), 6.84 (d, J=8.2 Hz, 1H), 4.08 (s, 1H). HRMS (ESI): m/z calcd. for C₁₄H₁₁ClN₂O₄ [M+H]⁺ 307.0486, Found 307.0479.

### Example 11: Synthesis of Compound (11)

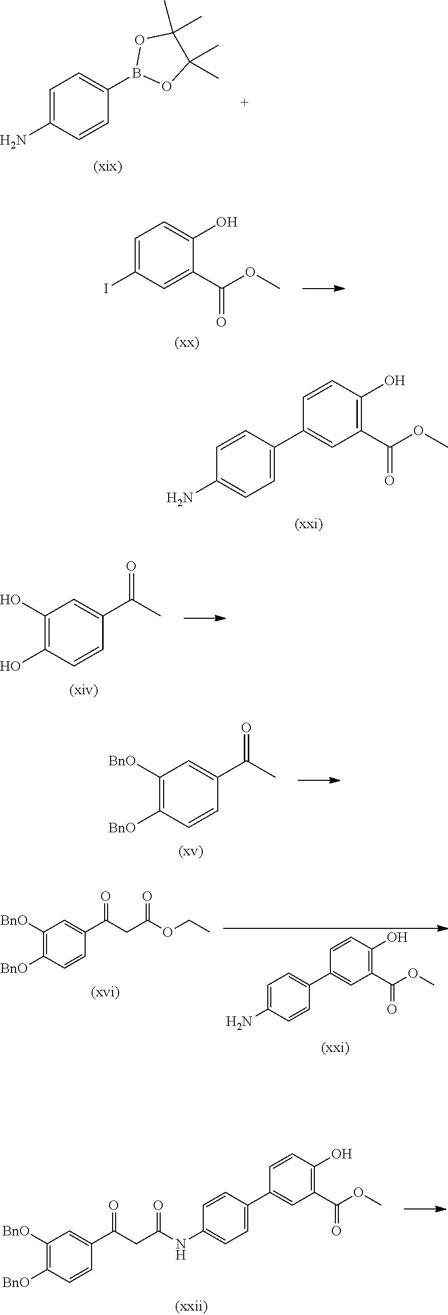

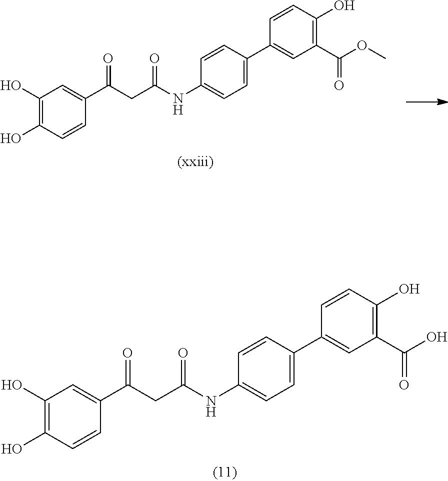 [SMILES: COC(=O)c1cc(-c2ccc(NC(=O)CC(=O)c3ccc(O)c(O)c3)cc2)ccc1O ; O=C(CC(=O)c1ccc(O)c(O)c1)Nc1ccc(-c2ccc(O)c(C(=O)O)c2)cc1]

In a pressure vessel, compound 7 (5.0 g, 22.8 mmol), compound 8 (7.6 g, 27.4 mmol), DME (5.0 mL per mmol of compound 7) and H₂O (2.50 mL per mmol of compound 7) were added. Air was then removed by vacuum and then the vessel was purged with nitrogen. Pd(PPh₃)₄ (2.3 g, 2.74 mmol) and Na₂CO₃ (5.8 g, 54.8 mmol) were then added and oxygen was removed after each addition. The pressure vessel was sealed and the mixture was stirred at 75° C. overnight. The mixture was cooled at rt, water was added and the mixture was extracted with EtOAc. The combined organic layers were rinsed with brine, dried over MgSO4 and concentrated. The crude product was purified by column chromatography with dichloromethane as the eluent to afford compound 9.

A solution of Compound 11 (10.0 g, 23.7 mmol) in methanol (200 ml) was treated with 1 M aqueous sodium hydroxide (237 mL, 237 mmol) and the mixture was heated at reflux for 6 h. The mixture was cooled to rt and the methanol was removed under vacuum. The aqueous residue was treated with 2 M hydrochloric acid to pH ca. 1. The precipitate was collected by filtration, washed with water and dried under vacuum to give compound 12. Yield: 7.8 g (78.0%), ¹H NMR (400 MHz, DMSO) δ 11.34 (s, 1H), 10.24 (s, 1H), 9.98 (s, 1H), 9.42 (s, 1H), 8.02 (d, J=2.4 Hz, 1H), 7.82 (dd, J=8.6, 2.4 Hz, 1H), 7.67 (d, J=8.7 Hz, 1H), 7.59 (d, J=8.7 Hz, 1H), 7.43 (dd, J=8.3, 2.0 Hz, 1H), 7.40 (d, J=2.0 Hz, 1H), 7.05 (d, J=8.6 Hz, 1H), 6.85 (d, J=8.2 Hz, 1H), 4.01 (s, 1H). HRMS (ESI): m/z calcd. for C₂₂H₁₇NO₇ [M+H]⁺ 408.1083, Found 408.1079.

### Example 12: Inhibition of Amylin Fibril Formation

This example describes the analysis of representative compounds of Formula III, using the thioflavin-T assay to assess compound-mediated inhibition of amylin fibril formation.

### Methods

### Solubility and Spectral Analysis

Test compounds (1, 1A, 1B, 9) were dissolved with addition of 1M NaOH and were all 100% soluble in water at pH 11 or above, and were pale yellow when dissolved. The compounds were titrated down to pH 8-9 with 1 M HCl and solubility determined immediately, and following overnight and longer term storage. Compounds 1, 1A and 1B are samples of the same compound.

The absorbance across 400 nm to 550 nm of all compounds at pH 10.8 was determined using a UV-Vis spectrophotometer (Molecular Devices SpectraMax, data not shown).

### Thioflavin-T Assay

The ability of the compounds to inhibit human amylin (hA) fibril formation was assessed using the thioflavin-T assay, as described in J. F. Aitken et al., Suppression by polycyclic compounds of the conversion of human amylin into insoluble amylin amyloid., Biochem J., 374 (2003), 779-784. Inhibition was assessed at three hA:compound ratios (1:1, 1:0.1, 1:0.01) for each compound.

### Results

### Solubility and Spectral Analysis

Compounds 1, 1A and 1B remained 100% soluble during initial suspension and on titration and pH adjustment, while compound 9 was possibly slightly less soluble (95%) at pH 8-9. The compounds remained soluble overnight and on longer-term storage. The compounds were light sensitive when dissolved, and become progressively darker in colour with time.

All 4 compounds underwent a colour change with addition of 1M HCl, becoming either very pale yellow (1, 1A, 9) or colourless (1B).

Absorbance over a spectrum from 400 nm to 550 nm was determined for each compound at pH 10.8 (data not shown).

### Thioflavin-T Assay

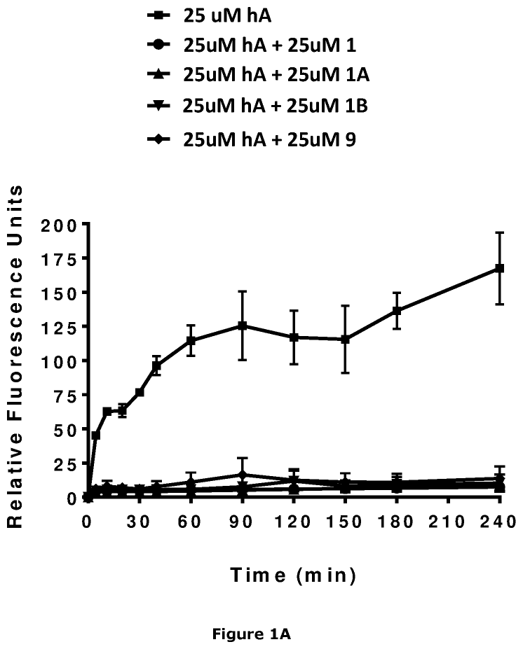

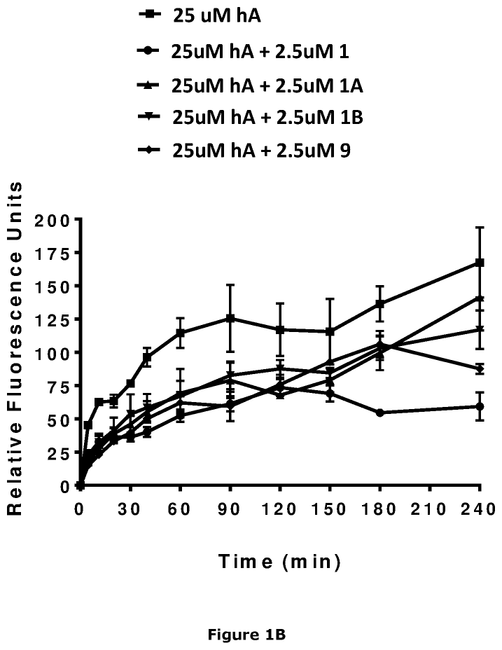

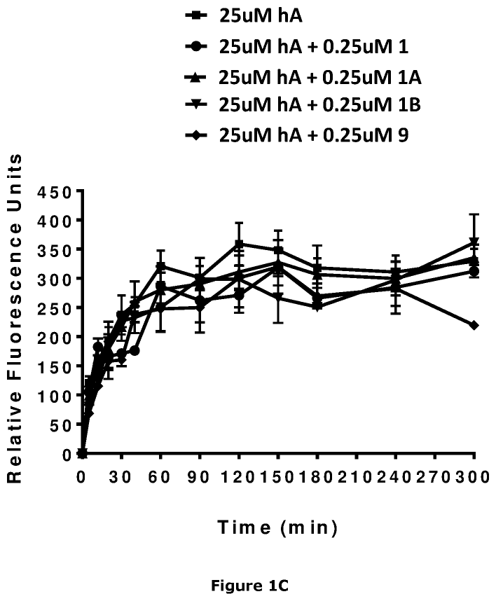

Inhibition of hA fibril formation was observed for each compound and fibril inhibition was dependent on the hA:compound ratio and on the initial concentration of hA. As can be seen in FIG. 1A, complete inhibition of fibril formation is observed for each compound at an hA:compound ratio of 1:1. Inhibition of hA fibril formation was observed at 1:0.1 hA:compound ratios as shown in FIG. 1B, particularly for compound 1, and to a lesser degree at 1:0.01 hA:compound ratios as shown in FIG. 1C.

The time-courses depicted in FIGS. 1A to 1C indicate that the compounds have greater inhibition of fibril formation over time if they prevent the initial nucleation events, even at lower concentrations of compounds.

### Example 13: Inhibition of Amylin-Evoked Cell Death

This example describes the analysis of representative compounds of Formula III, using cell death assays to assess compound-mediated inhibition of amylin-evoked cell death.

### Methods

The compounds 1, 1A, 1B and 9 were tested.

CM cells were cultured overnight in RPMI medium. Human amylin (hA) stock solutions (500 μM) were prepared freshly in water and diluted in medium to a final concentration of 10 μM. Aliquots of hA were then pre-mixed with various compounds at 1:1 and 1:10 molar ratio and incubated for 2 hrs at RT. The mixtures were then added to cells and incubation overnight for 16-20 hours. Untreated control cells were treated similarly, and treatment with Kp7-6 peptide was served as positive treatment control.

Apoptotic beta-cell death was measured using Cell Death Detection Elisa, as described in Zhang S., Diabetes, 57, 348-356, 2008; J. Biol. Chem., 278, 52810-52819, 2003, as provided in ROCHE Cell Death Detection ELISA plus (Cat No: 11774425001).

### Results

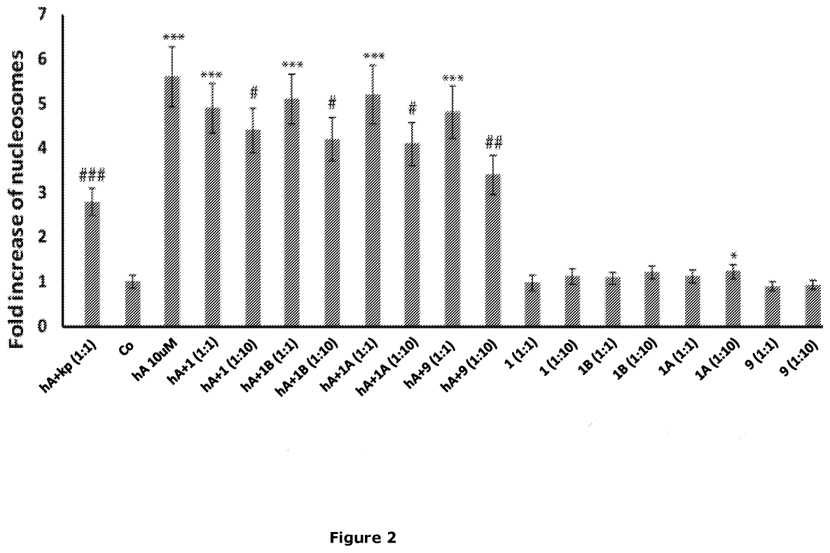

Results shown in FIG. 2 represent enrichment of nucleosomes in the sample (cell lysate), and are expressed as relative to controls (C₀) which were set at one. Values are mean±SE of four independent experiments, each performed in duplicate. * P<0.05; **P<0.01 versus control; ***P<0.001 versus control; #P<0.05 versus hA-treated cells.

As can clearly be seen in FIG. 2, statistically significant inhibition of amylin-evoked cell death was observed with each compound, at both 1:1 and 1:10 hA:compound molar ratios. Little or no compound-mediated cell death was observed (i.e., in the absence of amylin).

### Example 14: Inhibition of Amylin Fibril Formation

This example describes the analysis of representative compounds of Formulae II and III using the thioflavin-T assay to assess compound-mediated inhibition of amylin fibril formation.

### Methods

### Solubility and Physical Characteristics

Solutions of test compounds (2-9) were prepared as follows.
Compound 2: pale yellow solid. Mass 286.1. Add 1 M NaOH—only partially soluble approx. 50%. Leave 30 min, 100% soluble clear solution, pH 10.4. Add 1 M HCl, pH 9.4, still 100% soluble clear solution. Add 1 M HCl, pH 8.7, still 100% soluble clear solution. Still 100% soluble overnight.
Compound 3: Mass 406.1. Light yellow solid. Add 1 M NaOH—off-white/pale yellow solution 100% soluble pH 10.6. Add 1 M HCl, pH 9.3, slight colour change to clear solution, still 100% soluble. Add 1 M HCl, pH 8.4, clear solution, still 100% soluble. Still 100% soluble overnight.
Compound 4: green solid. Mass 270.1. Added 1 M NaOH—gold/orange colour partially soluble, leave 10 min—100% soluble pH 11.2. Added 1 M HCl, pH 9.2, 1 M HCl, pH 8.3, red colour, still 100% soluble. Overnight lost some solubility, to approx. 70% soluble.
Compound 5: Pale yellow solid. Mass 254.1. Add 1M NaOH. Partially soluble approx. 70%. Pale yellow colour, pH=11.3. Leave 30 min,—completely soluble. Add 1 M HCl, pH 8.9, slightly cloudy, some precipitation. Add 1 M NaOH—completely soluble pale yellow solution pH 10.4, but not soluble below pH 10.
Compound 6: gold colour solid. Mass 254.1. Completely insoluble pH 11.3 and pH 2.1.
Compound 7: off-white solid. Mass 298.1. Completely insoluble pH 11.1 and pH 1.8.
Compound 8: white solid. Mass 420.1. Added 1 M NaOH—partially soluble approx. 50%. Clear solution, leave 30 min—100% soluble pH 10.6. Added 1 M HCl, pH 8.4, still 100% soluble clear solution. Still 100% soluble overnight.
Compound 9: as described in Example 9 above.

### Thioflavin-T Assay

The ability of the compounds to inhibit human amylin (hA) fibril formation was assessed using the thioflavin-T assay, as described in J. F. Aitken et al., Suppression by polycyclic compounds of the conversion of human amylin into insoluble amylin amyloid., Biochem J 374 (2003) 779-784. Inhibition was assessed at three hA:compound ratios (1:1, 1:0.1, 1:0.01) for each compound.

### Results

### Thioflavin-T Assay

Inhibition of hA fibril formation was assessed for each compound, and at least a degree of inhibition was observed for each compound except for compound 8. Fibril inhibition was dependent on the hA:compound ratio and on the initial concentration of hA.

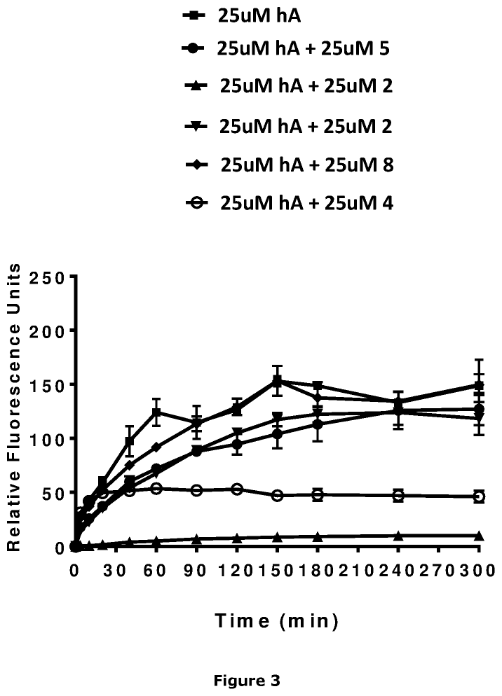

As can be seen in FIG. 3, complete inhibition of fibril formation is observed for compound 2 at an hA:compound ratio of 1:1 for the duration of the assay. Strong inhibition of fibril formation was also observed with compound 4:

Compounds 3, 5 and 8 showed little inhibition of fibril formation. For compounds 5 and 8, the observed lack of fibril inhibition is believed to be due to the lack of solubility at pH 7.4, being the pH at which the assay is carried out.

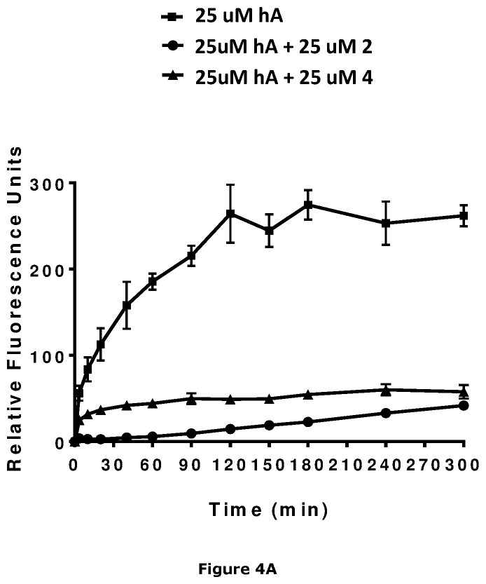

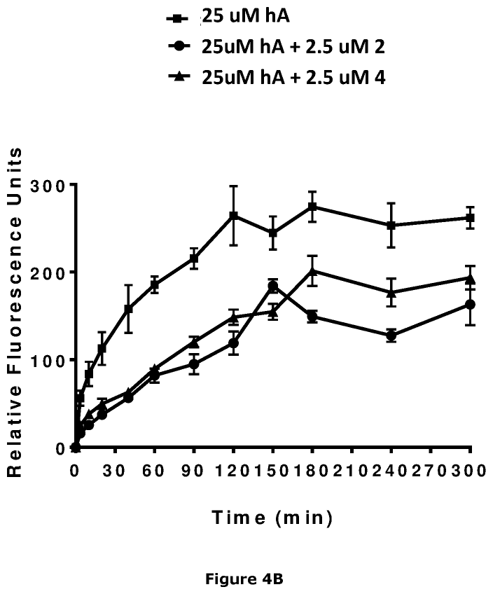

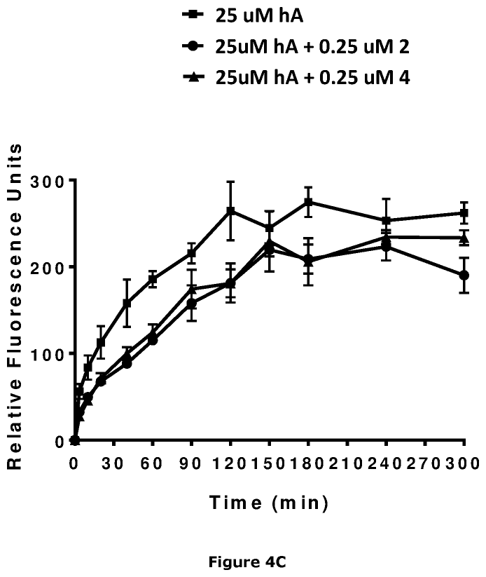

The time-courses depicted in FIGS. 4A to 4C indicate that the selected compounds 2 and 4 are able to inhibit fibril formation at higher hA:compound ratios, and have greater inhibition of fibril formation over time if they prevent the initial nucleation events, even at lower concentrations of compounds. Both compounds still inhibit amylin fibril formation at a ratio of hA: compound of 1:0.01.

### Example 15: Inhibition of Amylin-Evoked Cell Death

This example describes the analysis of representative compounds of Formulae II and III, using cell death assays to assess compound-mediated inhibition of amylin-evoked cell death.

### Methods

The compounds 1, 1A, 1B and 9 were tested.

RINm5F and CM cells were cultured and pre-incubated with test compounds at various concentrations for 1 h (1:1 and 1:10 mole ratio to human amylin) before addition of amylin. Amylin stock solutions were prepared freshly by dissolving lyophilized human amylin in water to 500 μM. Aliquots were then added to cultures to 10 μM (final) followed by incubation overnight for 16-20 hours. Untreated control cells were treated similarly, and treatment with Kp7-6 served as positive treatment control.

Apoptotic beta-cell death was measured using Cell Death Detection Elisa, as described above.

### Results

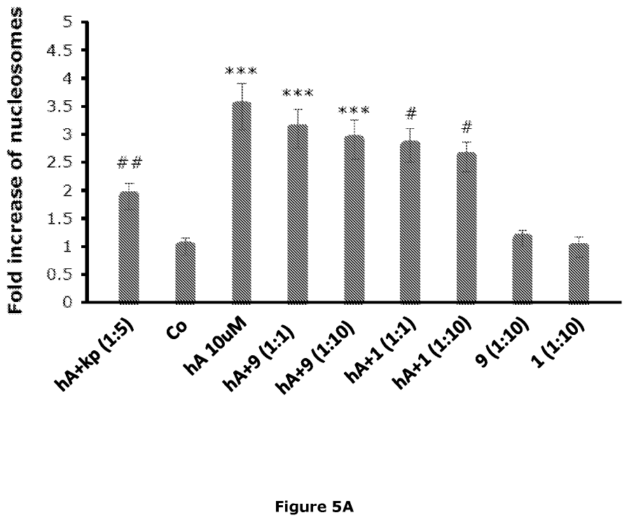

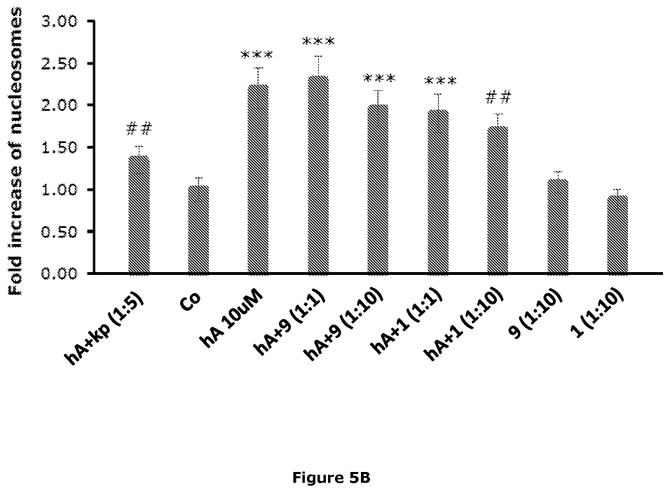

Results shown in FIG. 5 represent enrichment of nucleosomes in the sample (cell lysate), and are expressed as relative to controls (C₀) which were set at one. Values are mean SE of four independent experiments, each performed in duplicate. * P<0.05; **P<0.01 versus control; ***P<0.001 versus control; #P<0.05 versus hA-treated cells.

As can clearly be seen in FIG. 5A (CM cells) and FIG. 5B (RINm5F cells), statistically significant inhibition of amylin-evoked cell death was observed with each compound, at both 1:1 and 1:10 hA:compound molar ratios. Little or no compound-mediated cell death was observed (i.e., in the absence of amylin).

### Example 16: Inhibition of Amylin-Evoked Cell Death

This example describes the analysis of representative compounds described herein, using cell death assays to assess compound-mediated inhibition of amylin-evoked cell death.

### Methods

The compounds 1, 1A, 1B, 2, 4 and 9 were tested.

RINm5F and CM cells were cultured and pre-incubated with test compounds at various concentrations for 1 h (1:10 mole ratio to human amylin) before addition of amylin. Amylin stock solutions were prepared freshly by dissolving lyophilized human amylin in water to 500 μM. Aliquots were then added to cultures to 10 μM (final) followed by incubation overnight for 16-20 hours. Untreated control cells were treated similarly, and treatment with Kp7-6 served as positive treatment control.

Apoptotic beta-cell death was measured using the Cell Death Detection Elisa, as described above.

### Results

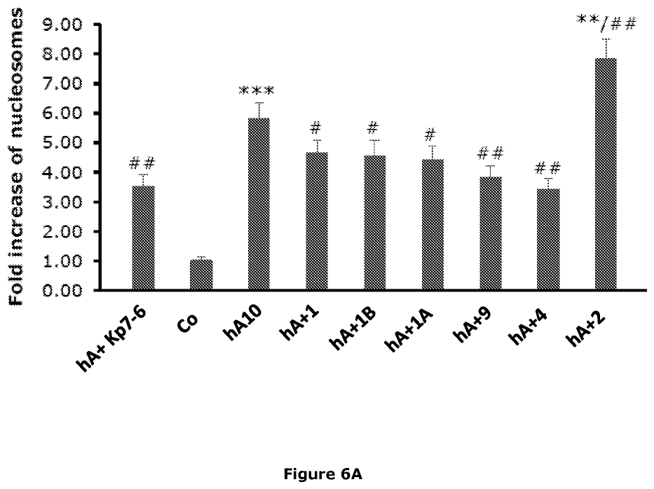

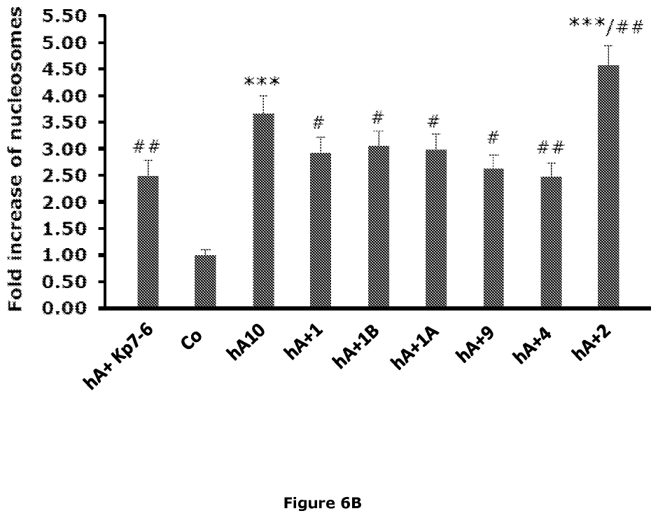

Results shown in FIG. 6 represent enrichment of nucleosomes in the sample (cell lysate), and are expressed as relative to controls (C₀) which were set at one. Values are mean±SE of four independent experiments, each performed in duplicate. * P<0.05; ***P<0.001 versus control; #P<0.05 versus hA-treated cells.

As can clearly be seen in FIG. 6A (CM cells) and FIG. 6B (RINm5F cells), statistically significant inhibition of amylin-evoked cell death was observed with these compounds, at both 1:10 hA:compound molar ratios. Little or no compound-mediated cell death was observed (i.e., in the absence of amylin) for all compounds other than compound 2.

### Example 17: Inhibition of Amylin-Evoked Cell Death

This example describes further analysis of representative compounds described herein, using cell death assays to assess compound-mediated inhibition of amylin-evoked cell death.

### Methods

The compounds 1, 4, 9, 10, 11, 14, 16, 18, 20, 21, 23 and 24 were tested. Rutin was used as a control.

RINm5F and CM cells were cultured overnight in RPMI medium (5% FBS). Human amylin (hA) stock solutions (500 μM) were prepared freshly in water and diluted in medium to a final concentration of 10 μM. Aliquots of hA (150 ul each) were then pre-mixed with various compounds at 1:10 molar ratio and incubated for 2 hrs at RT. The mixtures were then added to cells and incubated overnight for 16-18 hours. Untreated control cells were treated similarly with water.

Apoptotic beta-cell death was measured using the Cell Death Detection Elisa, as described above.

### Results

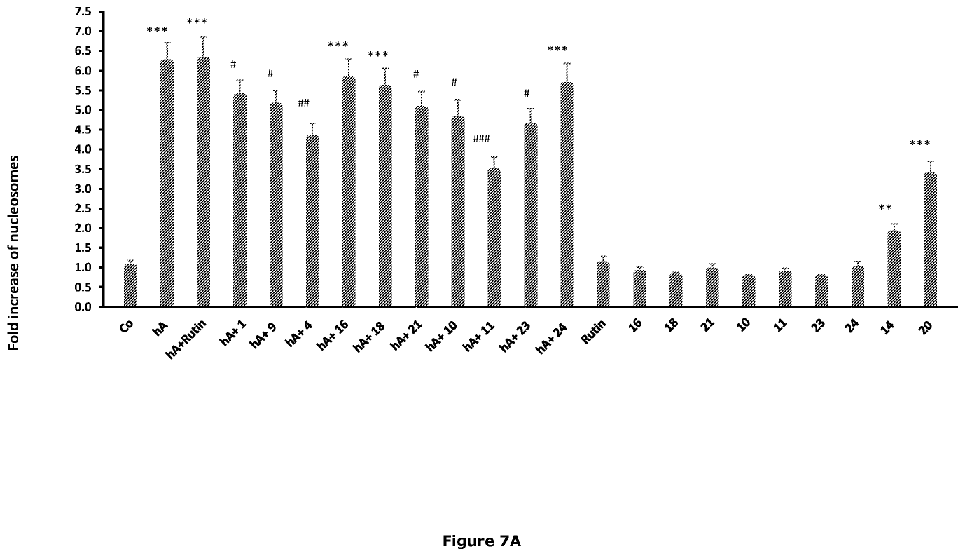

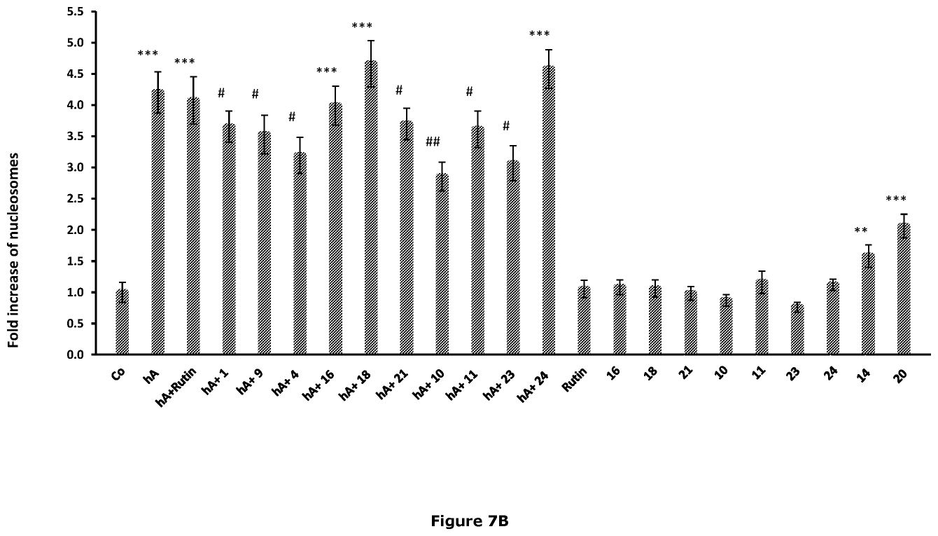

Results shown in FIG. 7 represent enrichment of nucleosomes in the sample (cell lysate), and are expressed as relative to controls (C₀) which were set at one. Values are mean±SE of three independent experiments, each performed in duplicate. * P<0.05; ***P<0.001 versus control; #P<0.05; ##P<0.01; ###P<0.001 versus hA-treated cells.

As can clearly be seen in FIG. 7A (CM cells) and FIG. 7B (RINm5F cells), statistically significant inhibition of amylin-evoked cell death was observed with a number of these compounds, at 1:10 hA:compound molar ratios. Little or no compound-mediated cell death was observed (i.e., in the absence of amylin) for all compounds, except compounds 14 and 20 which were cytotoxic to both CM and RIN cells.

### Conclusions

The aim of these experiments was to test the effect of compounds on human amylin misfolding and to test their efficacy in vitro in a human amylin transgenic mouse line, which overexpresses human amylin in the pancreatic Beta-cells.

Thioflavin-t fluorescence has shown that treatment with representative compounds (FIGS. 1, 3, 4) can suppress misfolding and aggregation of hA in vitro. Cell death assays (FIGS. 2, 5, 6, 7) show that representative compounds can suppress amylin-evoked apoptotic cell death.

These data provide evidence for the disaggregation of hA oligomers in presence of compounds as described herein. Without wishing to be bound by any theory, these results are consistent with the operation of a compound-mediated interaction with soluble hA oligomers, which is believed to prevent their conversion into cytotoxic structures and thus protects islet beta-cells from cell death.

### Example 18: Metabolism

This example describes the analysis of a representative compound described herein in an in vivo study of compound metabolism in an animal model.

### Methods

The in vivo metabolism of compound 4 was tested as follows.

Three adult male FVB/N non-transgenic mice were injected i.p with 0.3 mg compound ZBB03 41-1 per mouse. After 45 mins, animals were sacrificed and blood taken by cardiac puncture.

Serum samples prepared from these blood samples were metabolomically analysed to assess the stability and metabolic profile of the compound.

### Results

Metabolomic analysis showed that 45-70% of the parent compound 4 remained unchanged after 45 min. A single major metabolite, in which the ketone group was reduced to an alcohol, was observed in each of the mice. Notably, negligible amounts of other metabolites, including glucouronidated or methylated metabolites, were observed.

### Conclusions

The aim of this experiment was to assess the metabolic fate of the compound ZBB03 41-1 in vivo. In each sample, significant amounts of the unmetabolised compound 4 was observed, supporting the utility of this compound in the therapeutic methods described herein.

1. A compound of Formula II: 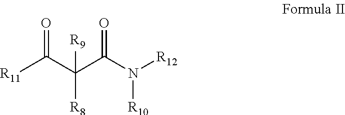
wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
R₁₁ is selected from optionally substituted aryl, optionally substituted heteroaryl, optionally substituted cycloalkyl, and optionally substituted heterocycloalkyl;
R₁₂ is selected from optionally substituted aryl, optionally substituted heteroaryl, optionally substituted cycloalkyl, and optionally substituted heterocycloalkyl;
or a pharmaceutically acceptable salt thereof.

2. A compound as claimed in claim 1 which is a compound of Formula IIa: 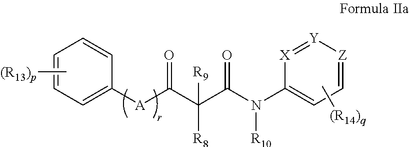
wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
each R₁₃ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
each R₁₄ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
A is selected from 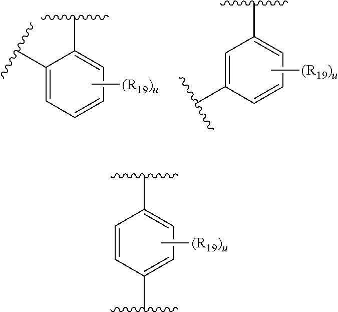
wherein each R₁₉ is independently selected from hydroxy, halide, carboxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, optionally substituted carboxyalkenyl; and
u is selected from 0, 1, 2, 3 and 4;
X is selected from C and N;
Y is selected from C and N;
Z is selected from C and N;
p is selected from 0, 1, 2, 3, 4 and 5;
q is selected from 0, 1, 2, 3 and 4;
r is selected from 0 and 1;
or a pharmaceutically acceptable salt thereof.

3. A compound as claimed in claim 1 which is a compound of Formula IIb: 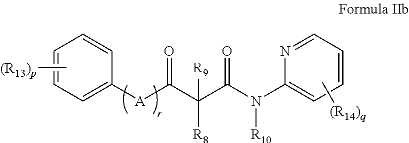
wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
each R₁₃ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
each R₁₄ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
A is selected from 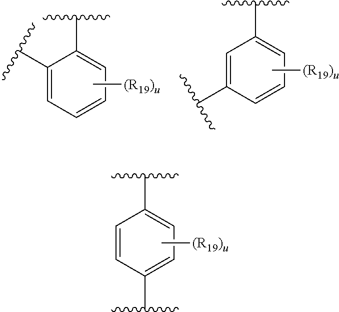
wherein each R₁₉ is independently selected from hydroxy, halide, carboxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
u is selected from 0, 1, 2, 3 and 4;
p is selected from 0, 1, 2, 3, 4 and 5;
q is selected from 0, 1, 2, 3 and 4;
r is selected from 0 and 1;
or a pharmaceutically acceptable salt thereof.

4. A compound as claimed in claim 1 which is a compound of Formula IIc: 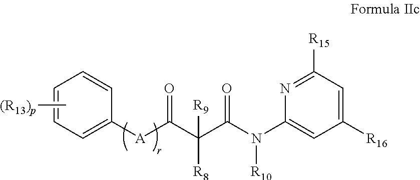
wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
each R₁₃ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₅ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₆ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
A is selected from 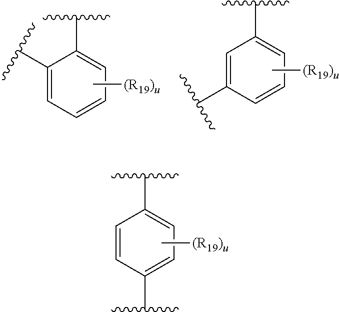
wherein each R₁₉ is independently selected from hydroxy, halide, carboxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
u is selected from 0, 1, 2, 3 and 4;
p is selected from 0, 1, 2, 3, 4 and 5;
r is selected from 0 and 1;
or a pharmaceutically acceptable salt thereof.

5. A compound as claimed in claim 1 which is a compound of Formula IId: 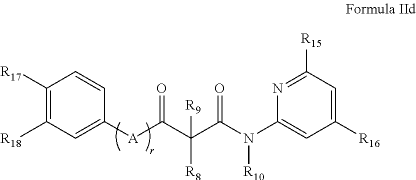
wherein:
R₈ is selected from hydrogen, optionally substituted C₁₋₄alkyl, and optionally substituted C₂₋₄alkenyl;
R₉ is selected from hydrogen, optionally substituted C₁₋₄alkyl, and optionally substituted C₂₋₄alkenyl;
R₁₀ is selected from hydrogen, optionally substituted C₁₋₄alkyl, and optionally substituted C₂₋₄alkenyl;
R₁₅ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₆ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₇ is selected from hydroxy, carboxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₁₈ is selected from hydroxy, halide, carboxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
A is selected from 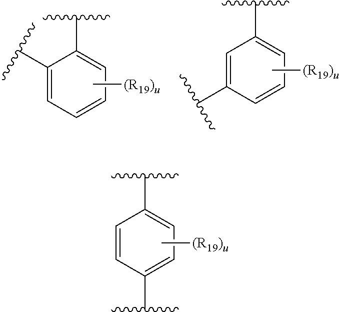
wherein each R₁₉ is independently selected from hydroxy, halide, carboxy, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
u is selected from 0, 1, 2, 3 and 4;
r is selected from 0 and 1;
or a pharmaceutically acceptable salt thereof.

6. A compound as claimed in claim 1 wherein:
R₈ is hydrogen;
R₉ is hydrogen;
R₁₀ is hydrogen;
R₁₁ is optionally substituted arylaryl;
R₁₂ is optionally substituted heteroaryl;
R₁₃ is carboxy, alkyloxy, halide or hydroxy;
R₁₄ is alkyloxy or halide;
A is phen-1,4-yl;
u is 0;
X is N, and Y and Z are C;
R₁₅ is alkyloxy or halide;
R₁₆ is alkyloxy or halide;
R₁₇ is carboxy, alkyloxy, hydroxyl or halide; and/or
R₁₈ is carboxy, alkyloxy, hydroxyl or halide.

7. A compound as claimed in claim 1 selected from the group comprising:

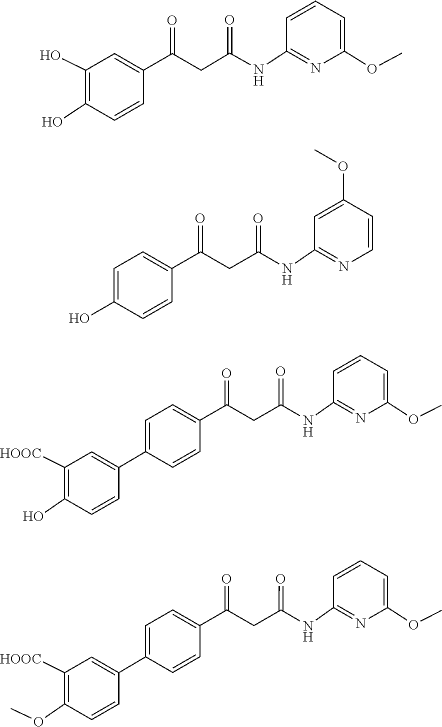

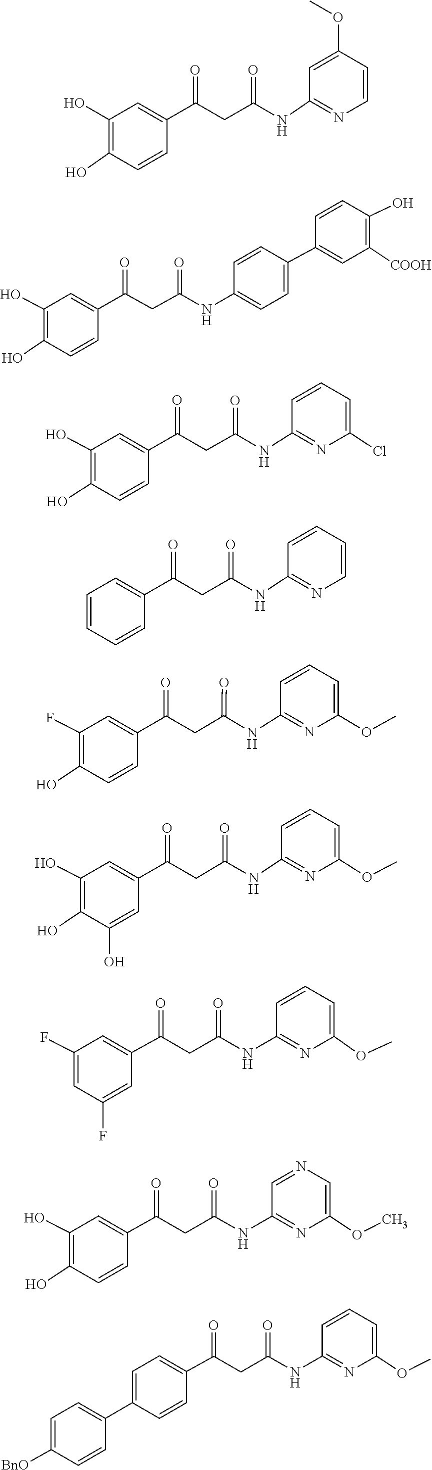

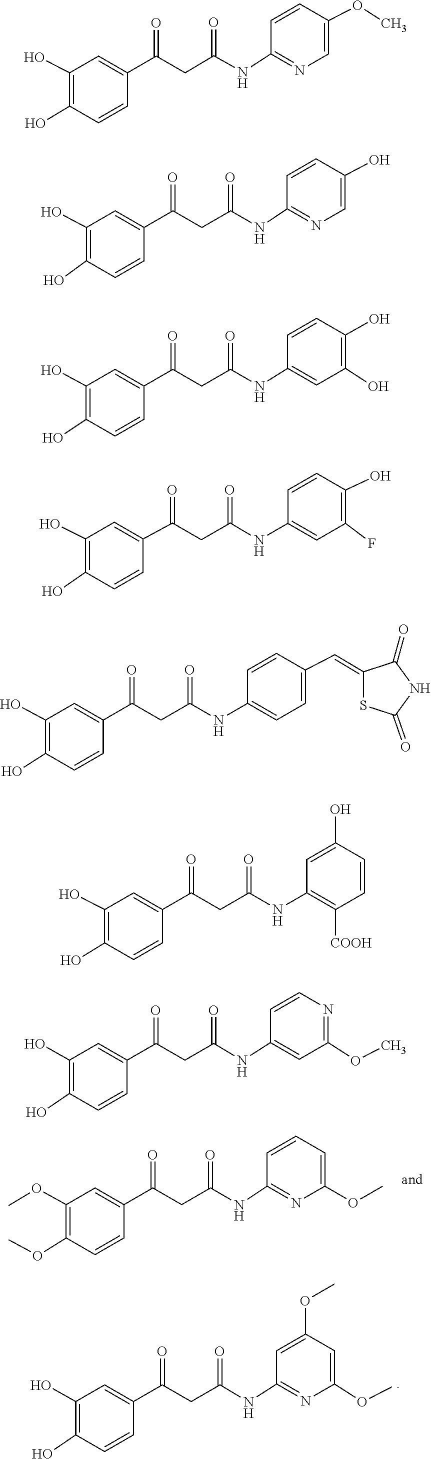

8. A compound of Formula I: 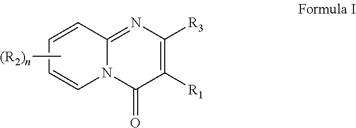
wherein:
R₁ is selected from hydrogen, halide, optionally substituted C₁₋₄alkyl, and optionally substituted C₂₋₄alkenyl;
each R₂ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₃ is selected from optionally substituted aryl, optionally substituted heteroaryl, optionally substituted heterocycloalkyl, and optionally substituted cycloalkyl;
n is selected from 0, 1, 2, 3 and 4;
or a pharmaceutically acceptable salt thereof.

9. A compound as claimed in claim 8 which is a compound of Formula Ia: 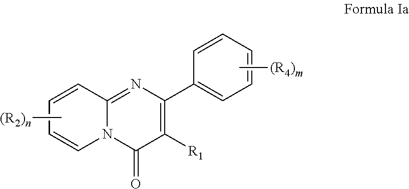
wherein
R₁ is selected from hydrogen, optionally substituted C₁₋₄alkyl, and optionally substituted C₂₋₄alkenyl;
each R₂ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
each R₄ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
n is selected from 0, 1, 2, 3 and 4;
m is selected from 0, 1, 2, 3, 4 and 5;
or a pharmaceutically acceptable salt thereof.

10. A compound as claimed in claim 8 which is a compound of Formula Ib: 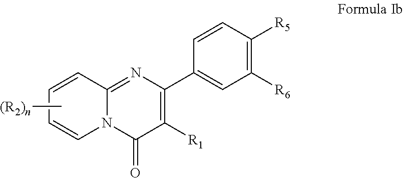
wherein:
R₁ is selected from hydrogen, optionally substituted C₁₋₄alkyl, and optionally substituted C₂₋₄alkenyl;
each R₂ is independently selected from hydroxy, halide optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₅ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₆ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
n is selected from 0, 1, 2, 3 and 4;
or a pharmaceutically acceptable salt thereof.

11. A compound as claimed in claim 8 which is a compound of Formula Ic: 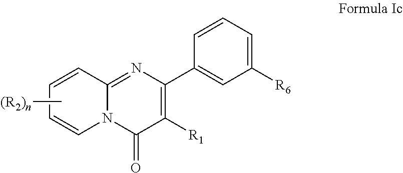
wherein
R₁ is selected from hydrogen, optionally substituted C₁₋₄alkyl, and optionally substituted C₂₋₄alkenyl;
each R₂ is independently selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₆ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
n is selected from 0, 1, 2, 3 and 4;
or a pharmaceutically acceptable salt thereof.

12. A compound as claimed in claim 8 which is a compound of Formula Id: 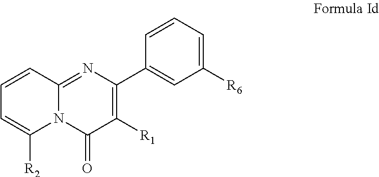
wherein
R₁ is selected from hydrogen, optionally substituted C₁₋₄alkyl, and optionally substituted C₂₋₄alkenyl;
R₂ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
R₆ is selected from hydroxy, halide, optionally substituted amino, optionally substituted aminoalkyl, optionally substituted aminoalkenyl, optionally substituted alkyloxy, optionally substituted alkenyloxy, optionally substituted alkyl, optionally substituted alkenyl, optionally substituted aryl, optionally substituted heteroaryl, optionally substituted heterocycloalkyl, optionally substituted cycloalkyl, carboxy, optionally substituted carboxyalkyl, and optionally substituted carboxyalkenyl;
or a pharmaceutically acceptable salt thereof.

13. A compound as claimed in claim 8 wherein:
R₁ is hydrogen;
each R₂ is independently selected from hydroxy and alkyloxy;
R₃ is selected from optionally substituted aryl;
each R₄ is independently selected from hydroxy and alkyloxy;
R₅ is selected from hydroxy and alkyloxy;
R₆ is selected from hydroxy and alkyloxy;
n is 1; and/or
m is 1 or 2.

14. A compound as claimed in claim 8 selected from the group comprising:

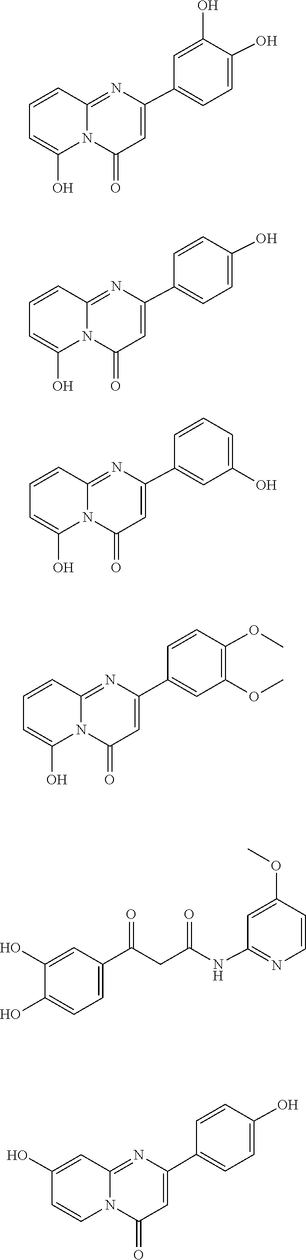 [SMILES: O=c1cc(-c2ccc(O)c(O)c2)nc2cccc(O)n12 ; O=c1cc(-c2ccc(O)cc2)nc2cccc(O)n12 ; O=c1cc(-c2cccc(O)c2)nc2cccc(O)n12 ; COc1ccc(-c2cc(=O)n3c(O)cccc3n2)cc1OC ; COc1ccnc(NC(=O)CC(=O)c2ccc(O)c(O)c2)c1 ; O=c1cc(-c2ccc(O)cc2)nc2cc(O)ccn12]

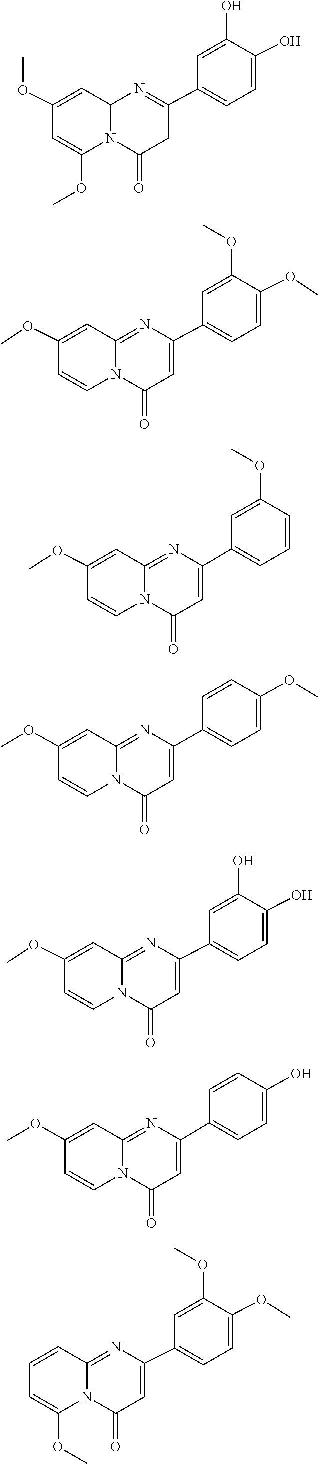

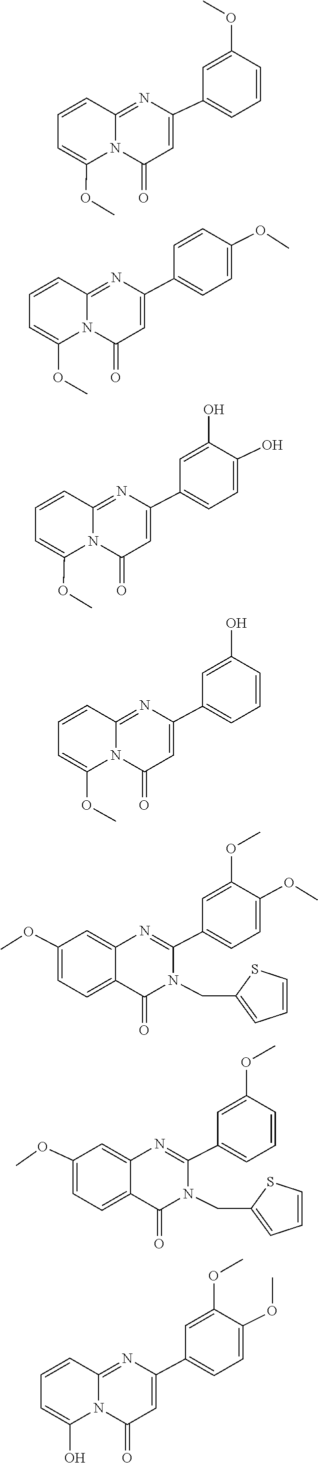

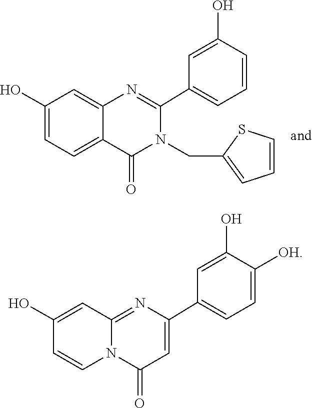

15. (canceled)

16. A method of treating or preventing diabetes, an amylin amyloid-associated disease, or islet of Langerhans beta-cell death, the method comprising administering to a subject in need thereof an effective amount of a compound of claim 1 or a pharmaceutically acceptable salt thereof.

17. A method of inhibiting, preventing, or reversing an amylin amyloidosis, or the formation of one or more amylin-amyloid fibrils or amylin amyloid plaques, the method comprising contacting the amylin amyloidosis or one or more amylin-amyloid fibrils or amylin amyloid plaques with an effective amount of a compound of claim 1 or a pharmaceutically acceptable salt thereof.

18. The method of claim 17 wherein the method comprises administering to a subject in need thereof an effective amount of the compound or pharmaceutically acceptable salt thereof.

19. The method of claim 17 wherein the amylin-amyloid fibrils are islet amylin-amyloid fibrils.

20. The method of claim 17 wherein the amylin-amyloid fibrils or amylin-amyloid plaques are present in islet of Langerhans beta-cells.

21. (canceled)
